(19) United States
(12) Patent Application Publication
(10) Pub. No.: US 2011/0268789 A1
(43) Pub. Date: Nov. 3, 2011

(54) USE OF PITUITARY ADENYLATE CYCLASE-ACTIVATING POLYPEPTIDE (PACAP) AND PACAP ANALOGS AS ADJUNCTIVE TREATMENTS WITH ANTICANCER AGENTS

(76) Inventors: Min Li, New Orleans, LA (US); Jerome L. Maderdrut, New Orleans, LA (US); David H. Coy, New Orleans, LA (US); Vecihi Batuman, New Orleans, LA (US)

(21) Appl. No.: 13/120,862

(22) PCT Filed: Sep. 25, 2009
(86) PCT No.: PCT/US09/58445

§ 371 Date: Jul. 20, 2011

Related U.S. Application Data
(60) Provisional application No. 61/194,166, filed on Sep. 25, 2008.

(51) Int. Cl. A61K 9/127; A61K 47/48; A61K 38/43; A61K 9/00; A61K 9/14; A61K 39/395; A61P 39/02; A61K 38/17; A61K 35/00

(52) U.S. Cl. 424/450; 514/21.3; 424/78.17; 424/94.3; 424/93.2; 424/499; 424/178.1; 424/400

(57) ABSTRACT

This invention relates to methods and compositions for the treatment, management or prevention of injuries to one or more of the organs of the body, such as the brain, heart, lung, kidneys, liver, and gastrointestinal tract, of humans or other mammals caused by one or more anticancer agents. The methods of this invention consist of the administration of an effective amount of one or more pituitary adenylate cyclase-activating polypeptide (PACAP)-like compounds, which includes native human PACAP38, native human PACAP27, native human vasoactive intestinal peptide (VIP), their agonists, analogs, fragments, and derivatives, with activities toward one or more of the PACAP/VIP receptors, including all of their various isoforms. This invention also provides pharmaceutical compositions of one or more PACAP-like compounds of the invention either alone or in combination with one or more other prophylactic/therapeutic agents useful for the treatment, management or prevention of injuries to the organs of the body of humans or other mammals undergoing cancer chemotherapy. Combination therapy with one or more PACAP-like compounds plus one or more anticancer agents can be used in the treatment of hematological cancers.

### FIELD OF THE INVENTION

This invention relates to methods and compositions for the treatment, management, or prevention of injuries to one or more of the organs of the body, such as the brain, heart, lung, kidneys, liver, and gastrointestinal tract, of humans or other mammals caused by one or more anticancer agents. The methods of this invention include the administration of an effective amount of one or more pituitary adenylate cyclase-activating polypeptide (PACAP)-like compounds, which includes native human PACAP38, human PACAP27, native human vasoactive intestinal peptide (VIP), their agonists, analogs, fragments, and derivatives, with activities toward one or more of the PACAP/VIP receptors, including all of their various isoforms. This invention also provides pharmaceutical compositions of one or more PACAP-like compounds of the invention either alone or in combination with one or more other prophylactic/therapeutic agents useful for the treatment, management or prevention of injuries to the organs of the body of humans or other mammals undergoing cancer chemotherapy. Combination therapy with one or more PACAP-like compounds plus one or more anticancer agents can be used in the treatment of, in particular, hematological cancers.

### BACKGROUND OF THE INVENTION

Cancer is the leading cause of death in industrialized countries. Chemotherapy is the preferred treatment for disseminated cancers and metastatic tumors. Chemotherapy is also frequently used when surgery or radiation therapy have not completely eradicated a localized tumor, or as an adjunctive treatment with surgery or radiation therapy. The maximal tolerable dose of the most commonly used cancer therapeutics is limited by their toxic effects on one or more major organs of the body of humans or other mammals. For example, the dose-limiting toxicity for cancer chemotherapy with cisplatin is nephrotoxicity (Kintzel, Drug Saf. 24:19-38, 2001), the dose-limiting toxicity for cancer chemotherapy with bleomycin is pulmonary toxicity (Chandler, Clin. Chest Med. 11:21-30, 1990), and the dose-limiting toxicity for cancer chemotherapy with doxorubicin is cardiotoxicity (Takemura et al., Prog. Cardiovasc. Dis. 49:330-352, 2007). Several strategies have been used to increase the maximal tolerable dose of cancer therapeutics, and thus increase their therapeutic effectiveness.

For example, cancer therapeutics have been conjugated to monoclonal antibodies directed against tumor-associated antigens (Wu et al., Nat. Biotechnol. 23:1137-1146, 2005) or to bioactive peptides whose receptors are highly expressed in selected types of tumors (Reubi, Endocr. Rev. 24:389-427, 2003) in order to preferentially deliver the anticancer agent to the interior of tumor cells. An alternate strategy to increase the efficacy of cancer therapeutics is to preferentially protect normal tissues against the cytotoxic effects of the anticancer agents (Hogle, Semin. Oncol. Nurs. 23:213-224, 2007).

Pituitary adenylate cyclase-activating polypeptide (PACAP) was isolated from ovine (sheep) hypothalami based on its ability to stimulate adenylate cyclase activity in rat anterior pituitary cell cultures (Miyata et al., Biochem. Biophys. Res. Commun. 164:567-574, 1989). PACAP exists as two α-amidated peptides with 38 (PACAP38; SEQ ID NO:1) or 27 (PACAP27; SEQ ID NO:2) amino acids. Both peptides have the same N-terminal 27 amino acids and are synthesized from the same prohormone. The sequence of PACAP38 is identical in all mammals and differs from the avian and amphibian orthologs by only one amino acid (Vaudry et al., Pharmacol. Rev. 52:269-324, 2000). PACAP is a member of the secretin/vasoactive intestinal peptide (VIP)/growth hormone-releasing hormone (GHRH) family, and PACAP27 has 68% sequence identity with VIP (SEQ ID NO:3). PACAP is most abundant in the brain and testis, but there are significant levels in other organs, including the pancreas, adrenals, thymus, spleen, lymph nodes, and duodenal mucosa (Vaudry et al., Pharmacol. Rev. 52:269-324, 2000). PACAP is synthesized as a preprohormone and is processed mainly by prohormone convertase 1, prohormone convertase 2 and prohormone convertase 4 (Li et al., Neuroendocrinology 69:217-226, 1999 and Li et al., Endocrinology 141:3723-3730, 2000). The half-life of [¹²⁵I]-PACAP38 in the bloodstream of rats following intravenous injection is 5-6 minutes (Banks et al., J. Pharmacol. Exp. Ther. 267:690-696, 1993). Members of the secretin/VIP/GHRH family are degraded in plasma mainly by aminodipeptidases, especially dipeptidyl peptidase IV (Zhu et al., J. Biol. Chem. 278:22418-2223, 2003).

A PACAP-specific receptor, designated as the PAC₁ receptor, has been cloned from several vertebrate species (Arimura, Jpn J. Physiol. 48:301-331, 1998 and Vaudry et al., 2000). It is a G-protein-coupled receptor with seven putative membrane-spanning domains and belongs to a family of glycoprotein receptors that are coupled to multiple signal transduction pathways (Segre et al., Trends Endocrinol. Metab. 4:309-314, 1993). PACAP binds not only to the PAC₁ receptor with a high affinity, but it also binds to the VIP1 (VPAC₁) and VIP2 (VPAC₂) receptors with an affinity comparable to or greater than VIP. On the other hand, VIP binds to the PAC₁ receptor with an affinity 1,000 times less than PACAP (Arimura, 1998). At least 10 splice variants of the rat PAC₁ receptor have been cloned and each variant is coupled to distinct combinations of signal transduction pathways (Vaudry et al., 2000). The second messengers include adenylate cyclase, phospholipase C, mitogen-activated protein (MAP) kinases, and calcium. PACAP/VIP receptor can be coupled to Gas and/or Gαi in different types of cells. PACAP/VIP receptors are expressed in many different types of normal and cancer cells, including the catecholamine-containing cells in the adrenal medulla and the sympathetic ganglia, microglia, astrocytes and some types of neurons in the central nervous system, and T- and B-lymphocytes, macrophages and dendritic cells in the immune system (Vaudry et al., Pharmacol. Rev. 52:269-324, 2000). PACAP is a potent stimulator of catecholamine secretion from the adrenal medulla (Watanabe et al., Am. J. Physiol. 269:E903-E909, 1995), and is a potent inhibitor of the secretion of tumor necrosis factor-α (TNF-α), interleukin (IL)-6 and IL-12 from activated macrophages (Ganea et al., Crit. Rev. Oral Biol. Med. 13:229-237, 2002). PACAP also stimulates the proliferation of C6 glioblastoma cells (Dufes et al., J. Mol. Neurosci. 21:91-102, 2003), AR4-2J pancreatic carcinoma cells (Buscail et al., Gastroenterology 103:1002-1008, 1992) and MCF-7 breast cancer cells (Leyton et al., Breast Cancer Res. Treat. 56:177-186, 1999), but inhibits the proliferation of HEL myeloid leukemia cells (Hayez et al., J. Neuroimmunol. 149:167-181, 2004), SW403 colonic adenocarcinoma cells (Lelièvre et al., Cell Signal. 10:13-26, 1998) and multiple myeloma cells (Li et al., Regul. Pept. 145:24-32, 2008; see FIG. 2).

Although PACAP was isolated during a screen for novel hypophysiotropic factors, it soon became apparent that it is a pleiotropic peptide (Arimura, Jpn J. Physiol. 48:301-331, 1998; Vaudry et al., Pharmacol. Rev. 52:269-324, 2000). Several laboratories investigated the extraordinarily potent neuroprotective/neurotrophic properties of PACAP shortly after its isolation. The cytoprotective effects of PACAP and VIP have been studied much more extensively in the nervous system than in any other major organ of the body. The cell types that were protected by PACAP in various in vitro models include cerebellar granule cells, dorsal root ganglion cells, sympathetic ganglion cells, mesencephalic dopaminergic neurons, and basal forebrain cholinergic neurons (Arimura, supra; Vaudry et al., supra). PACAP also prevented the neuronal death induced by gp120, the envelope glycoprotein of the human immunodeficiency virus (HIV), in rat hippocampal neuron/glia co-cultures. The dose-response curve was bimodal, with peaks at 10⁻¹³ M and 10⁻¹⁰ M (Arimura et al., Ann. N.Y. Acad. Sci. 739:228-243, 1994). The critical findings in this study have been confirmed by Kong et al. (Neuroscience 91:493-500, 1999), who used lipopolysaccharide (LPS) as the neurotoxin in primary murine cortical neuron/glia co-cultures. The neuroprotective effect at 10⁻¹² M was correlated with a significant reduction in the accumulation of nitrite in the culture medium. The neuroprotective effect of low (femtomolar) doses of PACAP in neuron/glia co-cultures was abolished by PD98059, a MAP kinase inhibitor, but the neuroprotective effect of high (nanomolar) doses of PACAP was not affected by PD98059 (Li et al., J. Mol. Neurosci. 27:91-106, 2005). However, the neuroprotective effect of nanomolar doses of PACAP was abolished by Rp-cAMP, a protein kinase A inhibitor.

The drawbacks of using peptides for neuroprotection in the brain include their poor transport across the blood-brain barrier and their short half-life in the circulation after systematic administration. However, PACAP38 has been shown to be transported from the blood to the brain via a saturable mechanism (Banks et al., J. Pharmacol. Exp. Ther. 267:690-696, 1993). Therefore, PACAP38 was tested as a neuroprotectant in common in vivo preclinical models of heart attack and stroke. Four-vessel occlusion in the rat was used to model the consequences of a heart attack for the brain (transient global forebrain ischemia). Blood flow to the forebrain was interrupted for 15 minutes (Uchida et al., Brain Res. 736:280-286, 1996). PACAP38 was administered intravenously at doses of 16 pmol/hr or 160 pmol/hr beginning 24 hours after the start of the four-vessel occlusion for 6 days using an implanted Alzet osmotic minipump. The PACAP-treated and vehicle-treated rats were sacrificed on day 7 post-occlusion, and the number of pyramidal cells in the CA1 field of the hippocampus was counted. Following the 15-minute occlusion, there was a significant reduction in the number of pyramidal cells in CA1 after 7 days in vehicle-infused rats. The reduction in the number of pyramidal cells at day 7 post-occlusion was significantly reversed in the rats continuously infused intravenously with PACAP38 at both doses (Uchida et al., supra). There were no obvious side-effects at these low doses in the continuously infused rats during the 6 days. Middle cerebral artery occlusion (MCAO) in the rat was used to model a stroke (transient focal cerebral ischemia). The middle cerebral artery was occluded for 2 hours using the intraluminal filament technique (Reglodi et al., Stroke 31:1411-1417, 2000). PACAP38 was administered intravenously at a dose of 160 pmol/hr beginning either 4, 8 or 12 hours after the start of the transient MCAO until 48-hours post-occlusion using an implanted Alzet osmotic minipump. The continuous intravenous infusion of PACAP38 beginning at 4, 8 or 12 hours after the start of the transient MCAO resulted in a reduction of the infarct volume of approximately 51%, 22% or 12%, respectively, 48 hours after the start of the MCAO. The reduction in the infarct volume when the treatment started 4 hours after the start of the MCAO was highly significant (P<0.01). The continuous intravenous infusion did not appear to alter plasma glucose levels, blood gases or blood pressure (Reglodi et al., supra). These observations suggest that small changes in the concentration of PACAP in the brain can alter the vulnerability of nerve cells to injury. PACAP has also been shown by other laboratories to be efficacious in other common in vivo preclinical models for neurodegenerative diseases, including spinal cord injury (Chen et al., Neurosci. Lett. 384:117-121, 2005) and Parkinson's disease (Reglodi et al., Behav. Brain Res. 151:303-312, 2004).

The neuroprotective effects of low concentrations of PACAP in the nervous system are indirect and are probably mediated by at least four distinct mechanisms. (1) PACAP is a potent anti-inflammatory peptide. It has been shown to inhibit the induction of inducible nitric oxide synthase (iNOS) in activated macrophages, to inhibit the production of the pro-inflammatory cytokines TNF-α, IL-6 and IL-12 in activated macrophages, and to stimulate the production of the anti-inflammatory cytokine IL-10 in activated macrophages (Ganea & Delgado, 2002). PACAP probably inhibits inflammation at multiple steps in the inflammatory cascade because it is an endogenous counter-regulator of the inflammatory process (Martinez et al., Proc. Natl. Acad. Sci. USA 99:1053-1058, 2002). PACAP is also an extraordinarily potent deactivator of activated microglial cells (Kong et al., 1999; Delgado et al., 2002), which are the resident macrophage-like cells in the nervous system. (2) Femtomolar (10⁻¹⁵ M) concentrations of PACAP increase the levels of the mRNA for activity-dependent neurotrophic factor in murine neuron/glia co-cultures (David et al., Society for Neuroscience (33rd Annual Meeting), New Orleans, La., # 38.1, 2003 (Abstract)). Furthermore, the number of PAC₁ receptors on reactive glial cells is increased following injury (Uchida et al., 1996). Brenneman et al. (Neuropeptides 36:271-280, 2002) had previously shown that femtomolar concentrations of PACAP stimulate the release of RANTES in astrocyte cultures and that immunoneutralization of RANTES reduces the neuroprotective effect of PACAP in neuron/glia co-cultures. (3) Yang et al. (J. Pharmacol. Exp. Ther. 319:595-603, 2006) have shown that femtomolar concentrations of PACAP inhibit microglial NADPH oxidase activity and extracellular superoxide levels in mesencephalic neuron/glia co-cultures. (4) Figiel & Engele (J. Neurosci. 20:3596-3605, 2000) have reported that PACAP increased the expression of the glutamate transporters GLT-1 and GLAST and increased the activity of the glutamate metabolizing enzyme glutamine synthetase in astrocytes. These effects of PACAP would be expected to decrease glutamatergic neurotransmission. Wu et al. (Neurobiol. Aging 27:377-386, 2006) reported that PACAP mRNA levels are reduced in the cortex in three different murine models of Alzheimer's disease.

Aubert et al. (2008) have reported that PACAP38 protects cerebellar granule cells in cell culture and in cerebellar slices against the toxic side-effects of the anticancer agent cisplatin. No in vivo experiments were reported in the article, presumably because a concentration as high as 10⁻⁷ M cannot be achieved with standard routes of parenteral administration because the transport of PACAP38 into the brain is saturable (Banks et al., 1993). In addition, cisplatin-induced cerebellar toxicity is not a clinically significant oncological problem. However, damage to the cerebellum is a clinically significant side-effect of cancer chemotherapy with cytarabine or 5-fluorouracil. The authors also claim that PACAP38 does not protect Chinese hamster ovary (CHO) cells, a tumor cell line, against the cytotoxic effects of cisplatin. This latter conclusion is based on a negative result using a nonhuman atypical ovarian cancer cell line with an ectopic transgene in an unknown location. In addition, Gupta (Biochem. Biophys. Res. Commun. 153:598-605, 1988) has shown that CHO cells have more than 10-fold higher activities of the multidrug resistant transporters than comparable human cells. CHO cells are commonly used for the recombinant production of mammalian proteins, but rarely (if ever) used as a cell line to study ovarian cancer. Most human ovarian cancer biopsy specimens express one or more PACAP/VIP receptors, but the parental CHO cell line does not express PAC₁, VPAC₁, or VPAC₂ receptors. The suggestion by Aubert et al. (2008) that PACAP would not protect solid tumors against cisplatin is not consistent with the relevant published literature and our new experiments (FIGS. 13 and 14). Oka et al. (Amer. J. Pathol. 155:1893-1900, 1999) reported that PACAP protects HP75 human pituitary adenoma cells against apoptotic cell death caused by treatment with transforming growth factor-β1, and PACAP has been shown more recently to protect PC-3 androgen-independent human prostate cancer cells (Gutiérrez-Cañas, I. et al. Br. J. Pharmacol. 139:1050-1058, 2003) and CRL-2768 rat schwannoma cells (Castorina, A. et al. Brain Res. 1241:29-35, 2008) against apoptotic cell death caused by serum withdrawal. More pertinent, Onoue et al. (FEBS J. 275:5542-5551, 2008) have shown that PACAP protects RIN-m5F insulinoma cells against apoptotic cell death caused by the anticancer agent streptozotocin. In addition, PACAP(6-38), a PACAP/VIP receptor antagonist, inhibited the growth in nude mice of xenografts of PC-3 human prostate cancer cells (Leyton et al., Cancer Lett 125:131-139, 1998), NCI-H838 human non-small cell lung cancer cells (Zia et al., Cancer Res 55:4886-4891, 1995) and MCF-7 human breast cancer cells (Leyton et al., 1999).

The cytoprotective properties of PACAP and VIP have been studied far less extensively in the kidney, heart, gastrointestinal tract, and lung than in the nervous system. PACAP has been shown to protect the kidney against injuries caused by ischemia/reperfusion (Riera et al., Transplantation 72:1217-1223, 2001; Szakaly et al., J. Mol. Neurosci. 36:89-96, 2008), the commonly used antibiotic gentamicin (Li et al., 2008) and light-chain immunoglobulin overload (Li et al., 2008). Nephrotoxicity is dose-limiting for many anticancer agents, including (but not limited to) cisplatin and carboplatin.

PACAP has also been shown to protect the heart (Sano et al., Regul. Pept. 109:107-113, 2002; Gasz et al., Peptides 27:87-94, 2006) and the small bowel of the gastrointestinal tract (Ferencz et al., J. Mol. Neurosci. 37:168-176, 2008) against ischemia/oxidative stress. VIP has been shown to protect the lung against injury caused by ischemia/cold storage (Alessandrini, Acta Biomed. Ateneo. Parmense. 65:59-73, 1994). The dose-limiting toxicity for cancer chemotherapy with doxorubicin is cardiotoxicity (Takemura & Fujiwara, 2007), while the dose-limiting toxicity for cancer chemotherapy with bleomycin is pulmonary toxicity (Chandler, 1990). Gastrointestinal side effects are very common with most anticancer agents.

The hepatoprotective properties of PACAP have not previously been systematically investigated. The dose-limiting toxicity for cancer chemotherapy with carmustine is usually myelosuppression, but hepatotoxicity or nephrotoxicity can sometimes limit the doses that can be used to treat some patients.

Native PACAP has already been administered to normal human volunteers by investigators in at least four different laboratories (Chiodera et al., Neuroendocrinology 64:242-246, 1996; Filipsson et al., J. Clin. Endocrinol. Metab. 82:3093-3098, 1997; Doberer et al., Eur. J. Clin. Invest. 37:665-672, 2007; Murck et al., Am. J. Physiol. 292:E853-E857, 2007) and to a patient with multiple myeloma under a U.S. FDA-approved protocol (Li et al., Peptides 28:1891-1895, 2007). The only untoward effect reported was a transient flushing.

The published literature indicates that PACAP-like peptides can protect neurons (neuroepithelial cells) against a very broad range of injuries, including ischemia/reperfusion injury. The published literature also indicates that PACAP-like peptides can protect renal, pulmonary and gastrointestinal epithelial cells against injury due to ischemia/reperfusion. Whether PACAP-like peptides can protect renal, pulmonary and gastrointestinal epithelial cells against any commonly used cancer chemotherapeutic has not been previously investigated. The published literature suggests that PACAP-like peptides promote the proliferation and survival of most epithelial cancer cells. Therefore, the published literature suggests that parenteral administration of PACAP-like peptides cannot be used as an adjunctive treatment with cancer chemotherapeutics for most solid epithelial tumors.

PACAP-like peptides have been shown to inhibit the proliferation of most normal hematopoietic cells (e.g., Ottaway et al., J. Immunol. 132:417-423, 1984; Boudard et al., J. Neurosci. Res. 29:29-41, 1991; Tatsuno et al., Endocrinology 128:728-734, 1991; Trejter et al., Histol. Histopathol. 16:155-158, 2001). PACAP-like peptides have also been shown to inhibit the proliferation of HEL myeloid leukemia cells (Hayez et al., 2004). PACAP-like peptides also inhibit the proliferation of multiple myeloma cells (Li et al., 2008).

Citation or discussion of a reference herein shall not be construed as an admission that such reference is prior art to the present invention.

### SUMMARY OF THE INVENTION

The inventors have found that native human PACAP38, native human PACAP27, native human vasoactive intestinal peptide (VIP), and their analogs, fragments, and derivatives, and including agonists of one or more of the PACAP/VIP receptors, are extremely effective in protecting the major organs of the body against injuries caused by commonly used cancer chemotherapeutics. Thus, the present inventors have determined that PACAP-like peptides and compounds can be used as cytoprotective adjunctive agents when administered to a subject being treated with a cancer chemotherapeutic.

Accordingly, the present invention relates to methods for the treatment, management, prevention, and inhibition of injuries to organs of the body, such as the brain, heart, lung, kidneys, liver, pancreas, gall bladder, gastrointestinal tract (e.g., the pharynx, esophagus, stomach, small intestine, large intestine, appendix, and colon), the adrenal gland, the thymus, the spleen, and the lymph nodes) of humans or other mammals caused by one or more anticancer agents. The method includes administering an effective amount of a PACAP-like compound, which includes native human PACAP38, native human PACAP27, VIP, their agonists, analogs, fragments, or derivatives, and which may have activities at one or more PACAP/VIP receptors, for the inhibition of a pathology-causing cell phenotype (e.g., a pathological condition resulting in injury to one or more organs of the body, such as the brain, heart, lung, kidneys, liver, pancreas, gall bladder, gastrointestinal tract (e.g., the pharynx, esophagus, stomach, small intestine, large intestine, appendix, and colon), the adrenal glands, the thymus, the spleen, and the lymph nodes) in a subject (e.g., a human or other mammal) treated with one or more anticancer agents. The anticancer agent may directly or indirectly cause injury to one or more organs of the body.

A PACAP-like compound may be administered to a subject before treatment, after treatment, or substantially simultaneously upon treatment, with one or more anticancer agents. In an embodiment, the subject has cancer (e.g., an epithelial cell cancer or a hematological cancer, such as multiple myeloma). In another embodiment of the invention, the PACAP-like compound is delivered directly or indirectly to an organ having a dose-limiting toxicity to the anticancer agent. In preferred embodiments, the organ is the brain, the heart, the lungs, the liver, the kidneys, the pancreas, gall bladder, the gastrointestinal tract (e.g., the pharynx, esophagus, stomach, small intestine (e.g., the duodenal mucosa), large intestine, appendix, and colon), the breasts, the ovaries, the testes, the adrenal glands, the thymus, the spleen, or the lymph nodes.

In another embodiment, the PACAP-like compound is administered one or more times to the subject at least 1, 2, 5, 10, 15, 20, 25, 30, 35, 40, 45, 50, or 55 minutes before or after treatment with one or more anticancer agents. In another embodiment, the PACAP-like compound is administered one or more times to the subject at least 1, 2, 5, 10, 15, 20, 24, 36, 48, or 60 hours before or after treatment with one or more anticancer agents. In yet another embodiment, the PACAP-like compound is administered one or more times to the subject at least 1, 2, 3, 4, 5, 6, 7, 8, 9, 10, 11, 12, 13, 14, 15, 16, 17, 18, 19, 20, 21, 22, 23, 24, 32, 36, 40, 44, 48, or 52 weeks before or after treatment with one or more anticancer agents.

For example, pulmonary toxicity usually limits the parenteral dose of bleomycin in subjects treated for testicular cancer. Accordingly, the method involves administering the PACAP-like compound (e.g., a PACAP-like peptide) to the lung as an aerosol in a subject treated for testicular cancer with bleomycin. In another example, when cancer chemotherapeutics with central nervous system or gastrointestinal dose-limiting toxicities are used, the PACAP-like cytoprotective adjunctive agents can be preferentially delivered to the brain or the gastrointestinal tract with intranasal or oral administration, respectively. Those skilled in the art will recognize that many other strategies can be used to deliver PACAP-like peptides to an organ of the body.

The inventors have also shown that PACAP-like compounds can have an additive anticancer effect with certain anticancer agents, particularly against hematopoietic or hematological cancer cells. Thus, in another embodiment of the invention, the PACAP-like compound may be administered to a subject being treated with one or more anticancer agents for a hematopoietic or hematological cancer, including lymphoid and myeloid cancers. In preferred embodiments, the hematopoietic or hematological cancer is a leukemia (e.g., lymphoid or myeloid leukemia, chronic myelogenous leukemia, or erythroleukemia), a lymphoma (e.g., Burkitt's lymphoma or mantle cell lymphoma), or a plasma cell dyscrasia (e.g., multiple myeloma or Waldenström's macroglobulinemia).

In certain embodiments, the PACAP-like compound may be a derivative of a naturally occurring PACAP peptide. In an embodiment, the derivative may be an N-acetyl derivative. In another embodiment, the derivative may be a propylamide derivative (e.g., a Lys₃₈-propylamide derivative). In yet another embodiment, the PACAP-like compound is linked to a polyethylene glycol polymer with a molecular weight from about 4 kilodaltons to about 40 kilodaltons. In yet another embodiment, the PACAP-like compound is flanked by amino acid consensus sequences for one or more proteolytic enzymes.

In certain embodiments, the PACAP-like compound may be an analog of a naturally occurring PACAP peptide. Maxadilan, a 61-amino-acid peptide with two disulfide bridges that is synthesized naturally in the salivary glands of the hematophagous sand fly Lutzomyia longipalpis, is an example of a conformational analog of PACAP according to the present invention. It has no obvious linear amino-acid sequence identities with PACAP but binds preferentially to the PAC₁ receptors with high affinity (Tatsuno, I. et al. Brain Res. 889:138-148, 2001; Lerner, E. A. et al. Peptides 28:1651-1654, 2007). The amino-acid sequences of maxadilan made by sand flies from different regions of Central and South America can differ by more than 20%. However, the relative positions of the cysteine residues in these bioactive orthologs are invariant and all of these bioactive orthologs have a similar predicted secondary structure. The amino-acid sequences of some naturally occurring maxadilans are described by Lanzaro et al. (Lanzaro, G. C. et al. Insect. Mol. Biol. 8:267-275, 1999). The amino-acid sequence of a naturally occurring maxadilan is shown as SEQ ID NO:70. Therefore, linear analogs of conformational analogs of PACAP, such as linear analogs of maxadilan (Reddy, V. B. et al. J. Biol. Chem. 281:16197-16201, 2006), would be expected to bind to and stimulate PACAP/VIP receptors. Those skilled in the art will recognize that additional conformational analogs of PACAP could be created by synthetic combinatorial chemistry or phage display technologies.

The PACAP-like compounds of this invention can be purified from normal cells or extracellular fluids, synthesized by the methods of recombinant molecular biology, or (in the most common embodiment) synthesized by the methods of peptide chemistry.

The efficacy of a PACAP-like compound, particularly in the protection and/or rescue of neurons, cardiomyocytes, hepatocytes, and lung, kidney and gastrointestinal epithelial cells, may depend on the concentration at which the compound is administered. The inventors have discovered that within the generally effective concentration range of the composition of this invention, there is a peak effectiveness, below which the effectiveness of the composition falls off to a significant degree. In a preferred embodiment, the concentration of the PACAP-like compound of the present invention is between about 10⁻¹³ M and about 10⁻⁶ M in either a cell culture medium or in the interstitial space or blood of a subject, which permits treatment of the subject with minimal risk of adverse side effects from the treatment (Reglodi et al., 2000; Li et al., 2007). In a preferred embodiment, the concentration of the PACAP-like compound is about 10⁻⁹ M. The present discovery makes possible the use of the composition of this invention in low concentrations to provide substantial protection and rescue of neurons, cardiomyocytes, hepatocytes, and lung, kidney and gastrointestinal epithelial cells. In a specific embodiment, the composition of the present invention protects these cells from injury or death.

In the methods of the present invention, injury to an organ may be due to treatment with one or more commonly used anticancer agents, including (but not limited to), e.g., cisplatin, carboplatin, oxaliplatin, bleomycin, mitomycin C, calicheamicins, maytansinoids, doxorubicin, idarubicin, daunorubicin, epirubicin, busulfan, carmustine, lomustine, semustine, thalidomide, lenalidomide, methotrexate, 6-mercaptopurine, fludarabine, 5-azacytidine, pentostatin (2′-deoxycoformycin), cytarabine (cytosine arabinoside), gemcitabine, 5-fluorouracil, hydroxyurea, etoposide, teniposide, topotecan, irinotecan, chlorambucil, cyclophosphamide, ifosfamide, melphalan, bortezomib, vincristine, vinblastine, vinorelbine, paclitaxel, and docetaxel.

In the methods of the present invention, the PACAP-like compound can be administered to the subject prior to or after administration of the anticancer agent to the subject. Alternatively, the PACAP-like compound and the anticancer agent may be administered to the subject substantially simultaneously (e.g., within 1 minute, 2 minutes, 5 minutes, 20 minutes, 1 hour, 2 hours, 5 hours, 1 day, 1 week, 1 month, or 1 year or more of each other). In one embodiment, the PACAP-like compound is administered after the anticancer agent has resulted in an injury to an organ. In another embodiment, the PACAP-like compound is administered before the anticancer agent has resulted in an injury to an organ.

The PACAP-like compounds of the present invention may be administered intravenously, intraperitoneally, subcutaneously, intramuscularly, or otherwise into the bloodstream in order to achieve the optimal concentration for the treatment, management, reduction, inhibition, or prevention of injuries to one or more of the organs of the subject caused by treatment with one or more anticancer agents. The PACAP-like compounds may be administered to the subject at a dose of between 1 μg to 1 gram (e.g., between 1 to 2500 μg). In a preferred embodiment, The PACAP-like compounds may be administered to the subject at a dose of between 100 to 1000 μg. In a more preferred embodiment, The PACAP-like compounds may be administered to the subject at a dose of about 500 μg.

The PACAP-like compounds of the present invention may be administered to the subject by intravenous infusion at a rate of between 1 pmol/kg body weight/hour to 1200 pmol/kg body weight/hour. In a preferred embodiment, the rate of intravenous infusion is between 1-100 pmol/kg body weight/hour. In another preferred embodiment, the rate of intravenous infusion is between 100-200 pmol/kg body weight/hour. In yet another preferred embodiment, the rate of intravenous infusion is between 200-600 pmol/kg body weight/hour. The intravenous infusion of the PACAP-like compound may be for between 1 to 12 hours or more (e.g., 24, 36, or 48 hours or more). Administration of the PACAP-like compound may be repeated one or more times over the course of an hour, a day, a week, a month, or a year (e.g., 2, 3, 4, 5, 6, 7, 8, 9, 10, 11, or 12 or more times). The intravenous administration of the composition of the present invention may be as a bolus injection, as a constant infusion or as a bolus injection followed immediately by a constant infusion. In a preferred embodiment, the subject is being treated with one or more chemotherapeutics for a hematological malignancy and the PACAP-like adjunctive agent is administered as a bolus injection (in order to saturate any serum binding proteins) followed immediately by a constant infusion.

The PACAP-like compounds of the present invention may be administered by inhalation or intranasally in order to have preferential access to the lung (Doberer et al., 2007) or the brain (Nonaka, N. et al. J. Pharmacol. Exp. Ther. 325:513-519, 2008), respectively.

The PACAP-like compounds of the present invention may be administered orally in a time-dependent (Gazzaniga, A. et al. Expert Opin. Drug Deliv. 3:583-597, 2006) or a pH-dependent (Gallardo, D. et al. Pharm. Dev. Technol. 13:413-423, 2008) formulation in order to have preferential access to different levels of the gastrointestinal tract or an injured region of the gastrointestinal tract, respectively.

The PACAP-like compounds of the present invention may be administered using viral vectors that code for one or more PACAP-like compounds that contain only some or all of the twenty amino acids that occur naturally in mammalian peptides.

The PACAP-like compounds of the present invention may be administered using cells that have been transfected with one or more polynucleotide sequences that code for one or more PACAP-like compounds that contain only some or all of the twenty amino acids that occur naturally in mammalian peptides.

The PACAP-like compounds of the present invention may be administered in a controlled-release (Kost, J. et al. Adv. Drug Deliv. Rev. 46:125-148, 2001) or a sustained-release (Hutchinson, F. G. et al. J. Control Release 13:279-294, 1990) formulation. In an embodiment, the subjects are administered one or more chemotherapeutic or anticancer agents as treatment for a hematological malignancy.

The PACAP-like compounds of the present invention may be administered after encapsulation in liposomes (Sethi, V. et al. Methods Enzymol. 391:377-395, 2005) or microparticles (Almeida, A. J. et al. Adv. Drug Deliv. Rev. 59:478-490, 2007).

The PACAP-like compounds of the present invention may be administered transcutaneously after encapsulation in dendrimers (Grayson, S. M et al. Chem. Rev. 101:3819-3868, 2001). In an embodiment, the subjects are treated with one or more chemotherapeutic or anticancer agents as treatment for a hematological malignancy.

The PACAP-like compounds of the present invention may be administered in combination with other cytoprotective adjunctive agents that have different mechanisms of action, such as amifostine, dexrazoxane, mesna, palifermin (human keratinocyte growth factor), and N-acetylcystein in order to have an additive or a synergistic effect.

The PACAP-like compounds of the present invention may be formulated in combination with a pharmaceutically acceptable carrier or excipient, or they may be formulated in a salt form, for administration to a subject (e.g., a human patient).

The PACAP-like compounds of the present invention may be used to treat, manage, reduce, inhibit, or prevent injuries to one or more organs of the body of humans or other mammals caused by both unconjugated anticancer agents and anticancer agents reversibly conjugated to a monoclonal antibody or to one or more bioactive peptides.

The PACAP-like compounds of the present invention may be used to reduce the incidence of delayed secondary cancers caused by one or more anticancer agents, especially the incidence of delayed secondary leukemias.

The composition of the present invention may be used to directly enhance the efficacy of anticancer agents in the treatment of certain cancer cells, e.g., hematopoietic or hematological cancer cells. The composition of the present invention may be used either in addition to a standard therapeutic regimen for a cancer, especially a hematopoietic or hematological cancer, or as a substitute for the glucocorticoid in one of the standard glucocorticoid-containing regimens, including (but not limited to) CHOP (cyclophosphamide, hydroxydaunorubicin, Oncovin, and prednisone), COP (cyclophosphamide, Oncovin and prednisone), COPP (cyclophosphamide, Oncovin, procarbazine, and prednisone), MOPP (mechlorethamine, Oncovin, procarbazine, and prednisone), and VAD (vincristine, Adriamycin and dexamethasone), for a cancer, especially a hematopoietic cancer.

### BRIEF DESCRIPTION OF THE DRAWINGS

FIG. 1 shows the primary amino acid sequences of PACAP38 (SEQ ID NO:1), PACAP27 (SEQ ID NO:2), VIP (SEQ ID NO:3), [D-Ser²]PACAP38 (SEQ ID NO:4), [Aib²]PACAP38 (SEQ ID NO:5), [D-Ser²,Lys³⁸-palmitoyl]PACAP38 (SEQ ID NO:6), [Aib²,Lys³⁸-palmitoyl]PACAP38 (SEQ ID NO:7), [Ala²²]PACAP38 (SEQ ID NO:8), [Ala¹⁶,Ala¹⁷,D-Lys³⁸]PACAP38 (SEQ ID NO:9), and [Lys³⁴]PACAP38 (SEQ ID NO:10). All of these compounds have been used in the experiments described in one or more of the figures listed below.

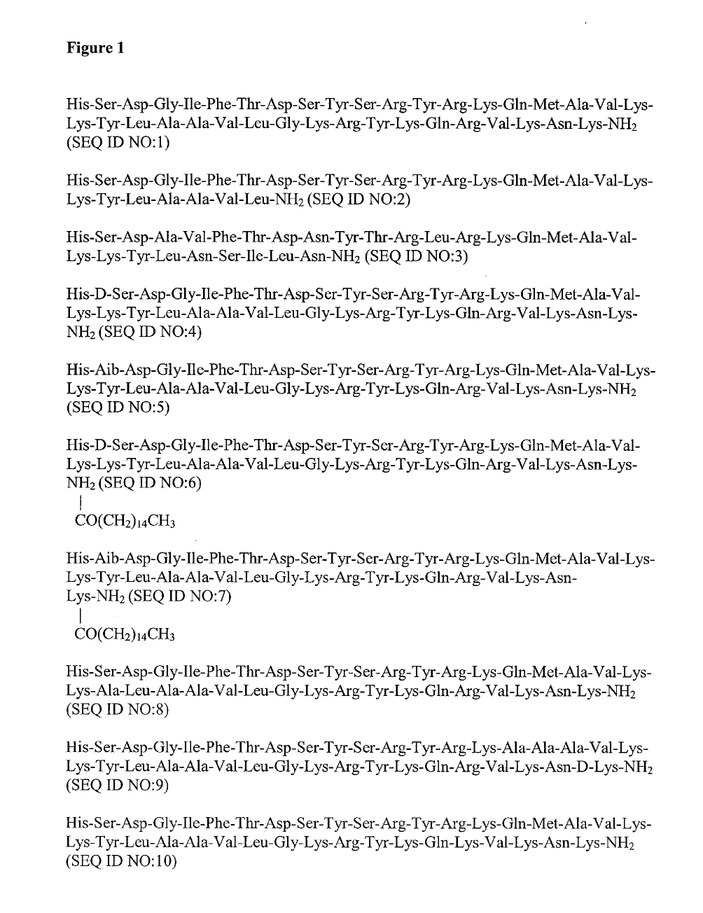

FIG. 2 shows the inhibitory effects of PACAP38 and PACAP38 analogs on the proliferation of light-chain immunoglobulin-secreting myeloma cells. The light-chain immunoglobulin-secreting human myeloma cells were cultured in RPMI 1640 medium supplemented with 10% non-inactivated fetal bovine serum and 0.05 mM 2-mercaptoethanol. The effects of PACAP38 and PACAP38 analogs on myeloma cell proliferation were assessed by determining incorporation bromodeoxyuridine into DNA during cell division. The number of myeloma cells approximately doubled during the 24-hour incubation period in the absence of treatment with PACAP-like peptides. Each value represents the mean plus/minus the standard error of four-eight determinations.

FIG. 3 shows the reduction in cisplatin-induced apoptotic cell death of human renal proximal tubule epithelial cells caused by varying concentrations of PACAP38. The HK-2 human kidney cells were cultured in Keratinocyte-Serum Free Medium supplemented with recombinant epidermal growth factor and bovine pituitary extract. The dose-dependent inhibitory effect of PACAP38 on apoptotic cell death was assessed by the quantitative determination of cytoplasmic histone-associated DNA-fragmentation (mono- and oligonucleosomes) after exposure to cisplatin for 24 hours. Each value represents the mean plus/minus the standard error of four determinations in three replicate experiments. **p<0.01 compared to the cisplatin-treated (control) cells.

FIG. 4 shows the reduction in cisplatin-induced apoptotic cell death of human renal proximal tubule epithelial cells caused by comparable concentrations of PACAP38 and [Aib²]PACAP38. The HK-2 human kidney cells were cultured in Keratinocyte-Serum Free Medium supplemented with recombinant epidermal growth factor and bovine pituitary extract. The dose-dependent inhibitory effect of PACAP38 on apoptotic cell death was assessed by the quantitative determination of cytoplasmic histone-associated DNA-fragmentation (mono- and oligonucleosomes) after exposure to cisplatin for 24 hours. Each value represents the mean plus/minus the standard deviation of four determinations in three different experiments. **p<0.01 and *p<0.05 compared to the cisplatin-treated (control) cells.

FIG. 5 shows the effect of PACAP38 on the adherence of human renal proximal tubule epithelial cells to extracellular matrix components in mice treated with cisplatin. The wells of a 96-well plate were coated with antibodies against fibronectin, collagen I or collagen IV. Treatment of HK-2 human kidney cells with 50 μM cisplatin significantly reduced the integrin-extracellular matrix interactions and decreased the cell surface-associated binding to fibronectin, collagen I and collagen IV as shown by the cell-based adhesion assay. PACAP38 partially reversed the decrease in binding to fibronectin and collagen IV in the cells exposed to cisplatin. Each value represents the mean plus/minus the standard error of four determinations. **p<0.01 compared to the corresponding cisplatin-treated (control) cells.

FIG. 6 shows the effects of PACAP38 on serum creatinine levels in mice treated with cisplatin. Male C57BL/6 mice were given a single intraperitoneal injection of 20 mg/kg of cisplatin. Twenty nanomoles of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. Each value represents the mean plus/minus the standard error of four determinations. ***p<0.001 and **p<0.01 compared to the group treated only with cisplatin.

FIG. 7 shows the effects of PACAP38 on blood urea nitrogen levels in mice treated with cisplatin. Male C57BL/6 mice were given a single intraperitoneal injection of 20 mg/kg of cisplatin. Twenty nanomoles of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. Each value represents the mean plus/minus the standard error of four determinations. ***p<0.001 and **p<0.01 compared to the group treated only with cisplatin.

FIG. 8 shows the effect of PACAP38 on the production of TNF-α production in the kidneys of mice treated with cisplatin. Male C57BL/6 mice were given a single intraperitoneal injection of 20 mg/kg of cisplatin. Twenty nanomoles of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. TNF-α was extracted from one kidney of each mouse and quantified with an enzyme-linked immunosorbent assay. Each value represents the mean plus/minus the standard error of four determinations. ***p<0.001 and **p<0.01 compared to the group treated only with cisplatin.

FIG. 9 shows the effects of PACAP38 on the morphology of the kidney in mice treated with cisplatin. Male C57BL/6 mice were given a single intraperitoneal injection of 20 mg/kg of cisplatin. Twenty nanomoles of PACAP38 were given intraperitoneally 1 hour before the injection of cisplatin and additional doses were given at 24 and 48 hours after the initial dose. The control group of mice were injected intraperitoneally with the same volume of saline as for the injections of cisplatin and PACAP38 on the same schedule. The mice were euthanized 24 hours after the final injection of PACAP38, and the kidneys were removed and fixed in 10% formalin. The fixed sections were stained with hematoxylin and eosin. The sections of the kidneys from control mice show intact renal tubular epithelial cells and well-preserved brush border membranes. The kidneys from mice treated with cisplatin had extensive tubular damage, tubular dilations, intratubular debris, and intratubular casts. Mice treated with both cisplatin and PACAP38 had relatively well preserved tubular morphology with less tubular damage and debris in the tubular lumens compared to cisplatin-treated mice. Tubular damage was more pronounced in the outer cortex in the cisplatin treated mice. The photomicrographs on the left side were made at a lower magnification (×160) than the photomicrographs on the right side (×320). The results described in FIGS. 5, 6, 7, and 8 were from the same experiment.

FIG. 10 shows the effects of PACAP38, [D-Ser²]PACAP38, [D-Ser²,Lys³⁸-palmitoyl]PACAP38, and VIP on serum creatinine levels in mice treated with cisplatin. Male C57BL/6 mice were given a single intraperitoneal injection of 20 mg/kg of cisplatin. Twenty nanomoles of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. Each value represents the mean plus/minus the standard error of six determinations. ***p<0.001 and **p<0.01 compared to the group treated only with cisplatin.

FIG. 11 shows the reduction in the doxorubicin-induced apoptotic cell death of human renal proximal tubule epithelial cells caused by comparable concentrations of PACAP38, [D-Ser²]PACAP38 and [Lys³⁴]PACAP38. The HK-2 human kidney cells were cultured in Keratinocyte-Serum Free Medium supplemented with recombinant epidermal growth factor and bovine pituitary extract. The dose-dependent inhibitory effects of PACAP38, [D-Ser²]PACAP38 and [Lys³⁴]PACAP38 on apoptotic cell death were assessed by the quantitative determination of cytoplasmic histone-associated DNA-fragmentation (mono- and oligonucleosomes) after exposure to cisplatin for 24 hours. Each value represents the mean plus/minus the standard deviation of four determinations in three replicate experiments. **p<0.01 and *p<0.05 compared to the doxorubicin-treated (control) cells.

FIG. 12 shows the reduction in the bleomycin-induced apoptotic cell death of human pulmonary epithelial cells caused by comparable concentrations of PACAP38, [D-Ser²]PACAP38 and [Lys³⁴]PACAP38. The L-132 human lung cells were cultured in Eagle's Minimum Essential Medium supplemented with 10% fetal bovine serum. The dose-dependent inhibitory effects of PACAP38, [D-Ser²]PACAP38 and [Lys³⁴]PACAP38 on apoptotic cell death was assessed by the quantitative determination of cytoplasmic histone-associated DNA-fragmentation (mono- and oligonucleosomes) after exposure to cisplatin for 24 hours. Each value represents the mean plus/minus the standard deviation of four determinations in three replicate experiments. **p<0.01 and *p<0.05 compared to the bleomycin-treated (control) cells.

FIG. 13 shows the reduction in the cisplatin-induced apoptotic cell death of pheochromocytoma cells caused by comparable concentrations of PACAP38, [D-Ser²]PACAP38 and [Lys³⁴]PACAP38. The PC-12 rat pheochromocytoma cells were cultured in F-12K medium supplemented with 15% horse serum and 2.5% fetal bovine serum. The dose-dependent inhibitory effects of PACAP38 and the PACAP38 analogs on apoptotic cell death were assessed by the quantitative determination of cytoplasmic histone-associated DNA-fragmentation (mono- and oligonucleosomes) after exposure to cisplatin for 24 hours. Each value represents the mean plus/minus the standard error of four determinations. **p<0.01 and *p<0.05 compared to the cisplatin-treated (control) cells.

FIG. 14 shows the reduction in the doxorubicin-induced apoptotic cell death of breast cancer cells caused by comparable concentrations of PACAP38 and N-acetyl[Ala^{16,17},D-Lys³⁸]PACAP38. The MCF-7 human breast cancer cells were cultured in Eagle's Minimum Essential Medium supplemented with 10% non-inactivated fetal bovine serum and 0.05 mM 2-mercaptoethanol. The effects of PACAP38 and N-acetyl[Ala^{16,17},D-Lys³⁸]PACAP38 on apoptotic cell death were assessed by the quantitative determination of cytoplasmic histone-associated DNA-fragmentation (mono- and oligonucleosomes) after exposure to doxorubicin for 48 hours. Each value represents the mean plus/minus the standard deviation of three determinations. **p<0.01 compared to the doxorubicin-treated (control) cells.

FIG. 15 shows the reduction in the etoposide-induced apoptotic cell death of leukemia cells by comparable concentrations of PACAP27 and PACAP38. The Jurkat human T-lymphocyte leukemia cells were cultured in RPMI 1640 medium supplemented with 10% non-inactivated fetal bovine serum. The effects of PACAP27 and PACAP38 on apoptotic cell death was assessed by the quantitative determination of cytoplasmic histone-associated DNA-fragmentation (mono- and oligonucleosomes) after exposure to etoposide for 48 hours. Each value represents the mean plus/minus the standard deviation of three determinations. **p<0.01 and *p<0.05 compared to the etoposide-treated (control) cells.

FIG. 16 shows the enhancement of doxorubicin-induced apoptotic cell death of light-chain immunoglobulin-secreting multiple myeloma cells caused by comparable concentrations of PACAP38 and [Ala¹⁶,Ala¹⁷,D-Lys³⁸]PACAP38. The light-chain immunoglobulin-secreting human myeloma cells were cultured in RPMI 1640 medium supplemented with 10% non-inactivated fetal bovine serum and 0.05 mM 2-mercaptoethanol. The effects of PACAP38, and [Ala^{16,17},D-Lys³⁸]PACAP38 on apoptotic cell death was assessed by the quantitative determination of cytoplasmic histone-associated DNA-fragmentation (mono- and oligonucleosomes) after exposure to doxorubicin for 48 hours. Each value represents the mean plus/minus the standard deviation of three determinations. **p<0.01 and *p<0.05 compared to the doxorubicin-treated (control) cells.

FIG. 17 shows the enhancement of carmustine-induced apoptotic cell death of light-chain immunoglobulin-secreting multiple myeloma cells caused by comparable concentrations of PACAP38 and [Ala²²]PACAP38. The light-chain immunoglobulin-secreting human myeloma cells were cultured in RPMI 1640 medium supplemented with 10% non-inactivated fetal bovine serum and 0.05 mM 2-mercaptoethanol. The effects of PACAP38, and [Ala²²]PACAP38 on apoptotic cell death was assessed by the quantitative determination of cytoplasmic histone-associated DNA-fragmentation (mono- and oligonucleosomes) after exposure to carmustine for 48 hours. Each value represents the mean plus/minus the standard deviation of three determinations. **p<0.01 and *p<0.05 compared to the carmustine-treated (control) cells.

FIG. 18 shows the enhancement of vincristine-induced apoptotic cell death of light-chain immunoglobulin-secreting multiple myeloma cells caused by various concentrations of PACAP38. The light-chain immunoglobulin-secreting human myeloma cells were cultured in RPMI 1640 medium supplemented with 10% non-inactivated fetal bovine serum and 0.05 mM 2-mercaptoethanol. The effects of PACAP38 on apoptotic cell death was assessed by the quantitative determination of cytoplasmic histone-associated DNA-fragmentation (mono- and oligonucleosomes) after exposure to vincristine for 48 hours. Each value represents the mean plus/minus the standard deviation of four determinations. **p<0.01 and *p<0.05 compared to the vincristine-treated (control) cells.

FIG. 19 shows the enhancement of thalidomide-induced apoptotic cell death of light-chain immunoglobulin-secreting multiple myeloma cells caused by various concentrations of PACAP38. The light-chain immunoglobulin-secreting human myeloma cells were cultured in RPMI 1640 medium supplemented with 10% non-inactivated fetal bovine serum and 0.05 mM 2-mercaptoethanol. The effects of PACAP38 on apoptotic cell death was assessed by the quantitative determination of cytoplasmic histone-associated DNA-fragmentation (mono- and oligonucleosomes) after exposure to thalidomide for 48 hours. Each value represents the mean plus/minus the standard deviation of four determinations. **p<0.01 and *p<0.05 compared to the thalidomide-treated (control) cells.

FIG. 20 shows the enhancement of thalidomide-induced apoptotic cell death of human erythroleukemia cells caused by comparable concentrations of PACAP38, [D-Ser²]PACAP38 and VIP. The human myeloid leukemia cells were cultured in RPMI 1640 medium supplemented with 10% non-inactivated fetal bovine serum. The effects of PACAP38, [D-Ser²]PACAP38 and VIP on apoptotic cell death was assessed by the quantitative determination of cytoplasmic histone-associated DNA-fragmentation (mono- and oligonucleosomes) after exposure to thalidomide for 48 hours. Each value represents the mean plus/minus the standard deviation of four determinations. **p<0.01 and *p<0.05 compared to the thalidomide-treated (control) cells.

### SEQUENCES

SEQ ID NO:1-NO:3 are the human sequences. SEQ ID NO:4-NO:66 are modifications of the corresponding human sequences. Below is a brief summary of the sequences presented in the accompanying sequence listing, which is incorporated by reference herein in its entirety:

SEQ ID NO:1 is the amino-acid sequence of PACAP38, which can be used according to the present invention.

SEQ ID NO:2 is the amino-acid sequence of PACAP27, which can be used according to the present invention.

SEQ ID NO:3 is the amino-acid sequence of VIP, which can be used according to the present invention.

SEQ ID NO:4 is the amino-acid sequence of [D-Ser²]PACAP38, which can be used according to the present invention.

SEQ ID NO:5 is the amino-acid sequence of [Aib²]PACAP38, which can be used according to the present invention.

SEQ ID NO:6 is the amino-acid sequence of [D-Ser²,Lys³⁸-palmitoyl]PACAP38, which can be used according to the present invention.

SEQ ID NO:7 is the amino-acid sequence of [Aib²,Lys³⁸-palmitoyl]PACAP38, which can be used according to the present invention.

SEQ ID NO:8 is the amino-acid sequence of [Ala²²]PACAP38, which can be used according to the present invention.

SEQ ID NO:9 is the amino-acid sequence of [Ala¹⁶,Ala¹⁷,D-Lys³⁸]PACAP38, which can be used according to the present invention.

SEQ ID NO:10 is the amino-acid sequence of [Lys³⁴]PACAP38, which can be used according to the present invention.

SEQ ID NO:11 is the amino-acid sequence of [Lys³⁸-palmitoyl]PACAP38, which can be used according to the present invention.

SEQ ID NO:12 is the amino-acid sequence of [D-Ser²,Ala¹⁶,Ala¹⁷,D-Lys³⁸]PACAP38, which can be used according to the present invention.

SEQ ID NO:13 is the amino-acid sequence of [Aib²,Ala¹⁶,Ala¹⁷,D-Lys³⁸]PACAP38, which can be used according to the present invention.

SEQ ID NO:14 is the amino-acid sequence of [D-Ala²]PACAP38, which can be used according to the present invention.

SEQ ID NO:15 is the amino-acid sequence of [D-Ser²,Nle¹⁷]PACAP38, which can be used according to the present invention.

SEQ ID NO:16 is the amino-acid sequence of [Aib²,Nle¹⁷]PACAP38, which can be used according to the present invention.

SEQ ID NO:17 is the amino-acid sequence of [D-Ala²,Nle¹⁷]PACAP38, which can be used according to the present invention.

SEQ ID NO:18 is the amino-acid sequence of [D-Ser²,Ala¹⁷]PACAP38, which can be used according to the present invention.

SEQ ID NO:19 is the amino-acid sequence of [Aib²,Ala¹⁷]PACAP38, which can be used according to the present invention.

SEQ ID NO:20 is the amino-acid sequence of [D-Ala²,Ala¹⁷]PACAP38, which can be used according to the present invention.

SEQ ID NO:21 is the amino-acid sequence of [Lys³⁶-palmitoyl]PACAP38, which can be used according to the present invention.

SEQ ID NO:22 is the amino-acid sequence of [Lys³²-palmitoyl]PACAP38, which can be used according to the present invention.

SEQ ID NO:23 is the amino-acid sequence of [Lys²⁹-palmitoyl]PACAP38, which can be used according to the present invention.

SEQ ID NO:24 is the amino-acid sequence of [D-Ser²,Lys³⁶-palmitoyl]PACAP38, which can be used according to the present invention.

SEQ ID NO:25 is the amino-acid sequence of [D-Ser²,Lys³²-palmitoyl]PACAP38, which can be used according to the present invention.

SEQ ID NO:26 is the amino-acid sequence of [D-Ser²,Lys²⁹-palmitoyl]PACAP38, which can be used according to the present invention.

SEQ ID NO:27 is the amino-acid sequence of [Aib²,Lys³⁶-palmitoyl]PACAP38, which can be used according to the present invention.

SEQ ID NO:28 is the amino-acid sequence of [Aib²,Lys³²-palmitoyl]PACAP38, which can be used according to the present invention.

SEQ ID NO:29 is the amino-acid sequence of [Aib²,Lys²⁹-palmitoyl]PACAP38, which can be used according to the present invention.

SEQ ID NO:30 is the amino-acid sequence of [Ala¹⁴]PACAP38, which can be used according to the present invention.

SEQ ID NO:31 is the amino-acid sequence of [Ala²⁰]PACAP38, which can be used according to the present invention.

SEQ ID NO:32 is the amino-acid sequence of [Ala²¹]PACAP38, which can be used according to the present invention.

SEQ ID NO:33 is the amino-acid sequence of [D-Ser²,Ala¹⁴]PACAP38, which can be used according to the present invention.

SEQ ID NO:34 is the amino-acid sequence of [D-Ser²,Ala²⁰]PACAP38, which can be used according to the present invention.

SEQ ID NO:35 is the amino-acid sequence of [D-Ser²,Ala²¹]PACAP38, which can be used according to the present invention.

SEQ ID NO:36 is the amino-acid sequence of [Ala¹⁴,Ala²⁰]PACAP38, which can be used according to the present invention.

SEQ ID NO:37 is the amino-acid sequence of [D-Ser²]PACAP27, which can be used according to the present invention.

SEQ ID NO:38 is the amino-acid sequence of [Aib²]PACAP27, which can be used according to the present invention.

SEQ ID NO:39 is the amino-acid sequence of [Ala²²]PACAP27, which can be used according to the present invention.

SEQ ID NO:40 is the amino-acid sequence of [D-Ala²]PACAP27, which can be used according to the present invention.

SEQ ID NO:41 is the amino-acid sequence of [D-Ser²,Nle¹⁷]PACAP27, which can be used according to the present invention.

SEQ ID NO:42 is the amino-acid sequence of [Aib²,Nle¹⁷]PACAP27, which can be used according to the present invention.

SEQ ID NO:43 is the amino-acid sequence of [D-Ala²,Nle¹⁷]PACAP27, which can be used according to the present invention.

SEQ ID NO:44 is the amino-acid sequence of [D-Ser²,Ala¹⁷]PACAP27, which can be used according to the present invention.

SEQ ID NO:45 is the amino-acid sequence of [Aib²,Ala¹⁷]PACAP27, which can be used according to the present invention.

SEQ ID NO:46 is the amino-acid sequence of [D-Ala²,Ala¹⁷]PACAP27, which can be used according to the present invention.

SEQ ID NO:47 is the amino-acid sequence of [D-Ser²,D-Leu²⁷]PACAP27, which can be used according to the present invention.

SEQ ID NO:48 is the amino-acid sequence of [Aib²,D-Leu²⁷]PACAP27, which can be used according to the present invention.

SEQ ID NO:49 is the amino-acid sequence of [Ala²²,D-Leu²⁷]PACAP27, which can be used according to the present invention.

SEQ ID NO:50 is the amino-acid sequence of [D-Ala²,D-Leu²⁷]PACAP27, which can be used according to the present invention.

SEQ ID NO:51 is the amino-acid sequence of [D-Ser²,Nle¹⁷,D-Leu²⁷]PACAP27, which can be used according to the present invention.

SEQ ID NO:52 is the amino-acid sequence of [Aib²,Nle¹⁷,D-Leu²⁷]PACAP27, which can be used according to the present invention.

SEQ ID NO:53 is the amino-acid sequence of [D-Ala²,Nle¹⁷,D-Leu²⁷]PACAP27, which can be used according to the present invention.

SEQ ID NO:54 is the amino-acid sequence of [D-Ser²,Ala¹⁷,D-Leu²⁷]PACAP27, which can be used according to the present invention.

SEQ ID NO:55 is the amino-acid sequence of [Aib²,Ala¹⁷,D-Leu²⁷]PACAP27, which can be used according to the present invention.

SEQ ID NO:56 is the amino-acid sequence of [D-Ala²,Ala¹⁷,D-Leu²⁷]PACAP27, which can be used according to the present invention.

SEQ ID NO:57 is the amino-acid sequence of [D-Ser²]VIP, which can be used according to the present invention.

SEQ ID NO:58 is the amino-acid sequence of [Aib²]VIP, which can be used according to the present invention.

SEQ ID NO:59 is the amino-acid sequence of [Ala²²]VIP, which can be used according to the present invention.

SEQ ID NO:60 is the amino-acid sequence of [D-Ala²]VIP, which can be used according to the present invention.

SEQ ID NO:61 is the amino-acid sequence of [D-Ser²,Nle¹⁷]VIP, which can be used according to the present invention.

SEQ ID NO:62 is the amino-acid sequence of [Aib²,Nle¹⁷]VIP, which can be used according to the present invention.

SEQ ID NO:63 is the amino-acid sequence of [D-Ala²,Nle¹⁷]VIP, which can be used according to the present invention.

SEQ ID NO:64 is the amino-acid sequence of [D-Ser²,Ala¹⁷]VIP, which can be used according to the present invention.

SEQ ID NO:65 is the amino-acid sequence of [Aib²,Ala¹⁷]VIP, which can be used according to the present invention.

SEQ ID NO:66 is the amino-acid sequence of [D-Ala²,Ala¹⁷]VIP, which can be used according to the present invention.

SEQ ID NO:67 is the amino-acid sequence of chicken (Galus domesticus) PACAP38, which can be used according to the present invention.

SEQ ID NO:68 is the amino-acid sequence of frog (Rana ridibunda) PACAP38, which can be used according to the present invention.

SEQ ID NO:69 is the amino-acid sequence of salmon (Oncorhynchus nerka) PACAP38, which can be used according to the present invention.

SEQ ID NO:70 is the amino-acid sequence of one naturally occurring variant of sand fly (Lutzomyia longipalpis) maxadilan, which can be used according to the present invention.

### DEFINITIONS

The following standard three-letter abbreviations are used herein to identify amino acid residues: Aib, α-aminoisobutyric acid; Ala, alanine; Arg, arginine; Asn, asparagine; Asp, aspartic acid; Cys, cysteine; Gln, glutamine; Glu, glutamic acid; Gly, glycine; His, histidine; Ile, isoleucine; Leu, leucine; Lys, lysine; Met, methionine; Nle, norleucine; Phe, phenylalanine; Pro, proline; Ser, serine; Thr, threonine; Trp, tryptophan; Tyr, tyrosine; Val, valine.

As used herein, the phrase “naturally occurring PACAP” peptide refers to pituitary adenylate cyclase-activating polypeptides (PACAP) that occur in nature and demonstrate the ability to promote adenylate cyclase activity and to bind to a PAC/VIP receptor. Examples of naturally occurring PACAP peptides include, but are not limited to, human PACAP27 (SEQ ID NO:2), human PACAP38 (SEQ ID NO:1), human VIP (SEQ ID NO:3), chicken PACAP38 (SEQ ID NO:67), frog PACAP38 (SEQ ID NO:68), salmon PACAP38 (SEQ ID NO:69), and sand fly maxadilan (SEQ ID NO:70).

As used herein, the phrase “PACAP-like compound” refers to a naturally occurring PACAP peptide and any peptide or peptidomimetic compound that is an ortholog, a paralog, an analog, a fragment, or a derivative thereof, or any compound that is a PACAP/VIP receptor agonist. Non-limiting examples of PACAP-like compounds are the polypeptides set forth in SEQ ID NOs: 1-70. PACAP-like compounds within the scope of the present invention exhibit substantial sequence identity, as that term is defined below, with one or more of the polypeptides set forth in SEQ ID NOs: 1-70. PACAP-like compounds may also be identified by the ability to bind to one or more of the PACAP/VIP receptors as an agonist (discussed below), the ability to promote an increase in the viability of, e.g., cisplatin-treated kidney epithelial cells (e.g., by at least 2%, 5%, 10%, 20%, 25%, 30%, or more, relative to kidney epithelial cells not treated with the PACAP-like compound), or the ability to promote a decrease in the rate of proliferation of, e.g., multiple myeloma cells (e.g., by at least 2%, 5%, 10%, 20%, 25%, 30%, or more, relative to multiple myeloma cells not treated with the PACAP-like compound).

As used herein, the phrase “PACAP/VIP receptor agonist” refers to any molecule, including a protein, naturally or synthetically post-translationally modified protein, polypeptide, naturally or synthetically modified polypeptide, peptide, naturally or synthetically modified peptide, and large or small nonpeptide molecule that binds to and stimulates one or more of the PACAP/VIP receptors.

The term “about” is used herein to mean a value that is ±10% of the recited value.

By “administration” or “administering” is meant a method of providing a dosage of an agent or composition of the invention to a mammal (e.g., a human), where the route is, e.g., topical, oral, parenteral (e.g., intravenous, intraperitoneal, intrarterial, intradermal, intramuscular, or subcutaneous injection, inhalation, optical drops, or implant), nasal, vaginal, rectal, or sublingual application in admixture with a pharmaceutically acceptable carrier adapted for such use. The preferred method of administration can vary depending on various factors, e.g., the components of the pharmaceutical composition, site of the potential or actual disease (e.g., the location of injured organ), and the severity of disease.

As used herein, the phrase “anticancer agent” refers to any compound, which is administered to a subject, preferably a human subject, to kill (e.g. by inducing apoptosis), slow the division of (e.g., by impairing mitosis), or otherwise mitigate the harmful effects of cancer cells in the subject. As used herein, an “anticancer agent” has the same meaning as a “chemotherapeutic,” a “chemotherapeutic agent,” and a “cancer therapeutic.” Particularly useful classes of anticancer agents include alkylating agents, antimetabolites, hormone agonists and antagonists, nitrosoureas, and plant alkaloids. Non-limiting examples of anticancer agents include cisplatin, carboplatin, oxaliplatin, bleomycin, mitomycin C, calicheamicins, maytansinoids, doxorubicin, idarubicin, daunorubicin, epirubicin, busulfan, carmustine, lomustine, semustine, thalidomide, lenalidomide, methotrexate, 6-mercaptopurine, fludarabine, 5-azacytidine, pentostatin, cytarabine, gemcitabine, 5-fluorouracil, hydroxyurea, etoposide, teniposide, topotecan, irinotecan, chlorambucil, cyclophosphamide, ifosfamide, melphalan, bortezomib, vincristine, vinblastine, vinorelbine, paclitaxel, and docetaxel, and derivatives and analogs thereof.

As used herein, the phrase “analog” refers to both conformational and linear sequence analogs. A peptide analog may contain one or more amino acids that occur naturally in mammalian cells but do not occur naturally in mammalian peptides. For example (but not by way of limitation), a peptide analog may contain γ-amino-N-butyric acid (GABA), β-alanine, ornithine, or citrulline. An analog of a peptide may also contain one or more nonnatural amino acids that do not occur naturally in mammalian cells. For example (but not by way of limitation), an analog of a peptide may also contain D-alanine, naphthylalanine, pyridylalanine, or norleucine. An analog may have an extension of one or more naturally occurring and/or nonnatural amino acids at its amino terminus and/or its carboxyl terminus. The extension at the amino terminus and/or the carboxyl terminus may include one or more additional copies of the same peptide and/or other bioactive peptides. The extension at the amino terminus and/or the carboxyl terminus may include one or more sites for proteolytic processing in order to make the extended peptide function as a precursor (prodrug) for the bioactive peptide. For example, the PACAP-like compounds may include cleavage sites at the amino terminus and/or the carboxyl terminus for one or more of the following proteolytic enzymes: trypsin, chymotrypsin, a prohormone convertase (e.g., prohormone convertase 1, 2, 4, or 7), furin, chymase, thrombin, calpain, a cathepsin (e.g., cathepsin A, B, D, G, H, or L), papain, Factor Xa, Factor IXa, Factor XIa, renin, chymosin, thermolysin, a kallikrein, an elastase, and a metalloproteinase.

As used herein, the phrase “peptidomimetic” refers to both hybrid peptide/organic molecules and nonpeptide organic molecules that have critical functional groups in a three-dimensional orientation that is functionally equivalent to the corresponding peptide (Marshall, G. R. Tetrahedron 49:3547-3558, 1993). Peptidomimetic compounds that are functionally equivalents to the PACAP-like compounds of the present invention can be rationally designed by those skilled in the art based on published structure-activity studies (e.g., Igarashi, H. et al. J. Pharmacol. Exp. Ther. 301:37-50, 2002; Igarashi, H. et al. J. Pharmacol. Exp. Ther. 303:445-460, 2002; Bourgault, S. et al. Peptides 29:919-932, 2008).

The phrases “percent identity” and “percent similarity” can be used to compare the amino-acid sequences of two peptides. To determine the percent identity of two amino acid sequences, the sequences are aligned for optimal comparison purposes (e.g., gaps can be introduced in the sequence of a first amino-acid sequence for optimal alignment with a second amino-acid sequence). The amino-acid residues at the corresponding amino-acid positions are then compared. When a position in the first sequence is occupied by the same amino-acid residue at the corresponding position in the second sequence, then the molecules are identical at that position. The percent identity between the two sequences is a function of the number of identical positions shared by the sequences (i.e., % identity=the number of identical overlapping positions/total number of positions×100%). In the most common embodiment, the two amino-acid sequences are the same length. To determine the percent similarity of two amino acid sequences, the sequences are also aligned for optimal comparison purposes. When a position in the first sequence is occupied by either the same amino-acid residue or a conserved amino acid at the corresponding position in the second sequence, then the molecules are similar at that position. The percent similarity between the two sequences is a function of the number of corresponding positions in the amino acid sequences at which the amino acids are either identical or the different amino acids are conserved substituents (i.e., % similarity=the number of identical or conserved overlapping positions/total number of positions×100%). A conservative substitution is a substitution of one amino acid by another amino acid with a similar side-chain. A conservative substitution frequently results in an analog with similar physical and biological properties. The following is a list of commonly defined classes of similar amino acids that occur naturally in mammalian peptides:

Aromatic side-chain: phenylalanine≅tyrosine≅tryptophan≅histidine;

Acidic side-chain: aspartic acid≅glutamic acid;

Basic side-chain: arginine≅lysine≅histidine;

β-Branched side-chain: threonine≅valine≅isoleucine;

Nonpolar side-chain: glycine≅alanine≅valine≅leucine≅proline≅methionine≅phenylalanine≅tryptophan;

Uncharged polar side-chain: glycine≅asparagine≅glutamine≅serine≅threonine≅cysteine≅tyrosine.

Those skilled in the art will recognize that many amino acids that occur naturally in mammalian cells but do not occur naturally in mammalian peptides and many nonnatural amino acids that do not occur naturally in mammalian cells can be substituted conservatively for one or more of the amino acids that occur naturally in mammalian peptides. For example (but not by way of limitation), hydroxyproline, dehydroproline and pipecolic acid could be substituted conservatively for proline; sarcosine, dialkylglycine and α-aminocycloalkane carboxylic acid could be substituted conservatively for glycine; and α-aminoisobutyric acid, naphthylalanine and pyridylalanine could be substituted conservatively for alanine. Percent identity and percent similarity are determined after optimal alignment of the two sequences without or without the introduction of one or more gaps in one or both amino-acid sequences. There are many algorithms that are well known to those skilled in the art that can be used to determine the optimal alignment. In the most common embodiment, the two amino-acid sequences are the same length.

By “substantial sequence identity” or “substantially identical” is meant a peptide or polypeptide exhibiting at least 50%, preferably 60%, 70%, 75%, or 80%, more preferably 85%, 90%, 95%, 97%, and most preferably 99% identity to a reference amino acid sequence (e.g., one or more of the polypeptides of SEQ ID NOs: 1-70). The length of the comparison sequence will generally be at least 5 contiguous amino acids, preferably at least 10 contiguous amino acids, more preferably at least 15, 20, 25, 30, 35, 40, 45, 50, 55, or 60 or more contiguous amino acids, and most preferably the full-length amino acid sequence. Preferably, the sequence of the peptide of the invention is at least 40, 50, 60, 70, 80, 90, 95, 97, 99%, or 100% identical to the reference sequence (e.g., one or more of the polypeptides set forth in SEQ ID NOs.: 1-70). Sequence identity is typically measured using BLAST® (Basic Local Alignment Search Tool) or BLAST®2 with the default parameters specified therein (e.g., Altschul et al., J Mol Biol 215:403-410 (1990); and Tatiana et al., FEMS Microbiol Lett 174:247-250 (1999)). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine. As used herein, the phrase “fragment” in the context of PACAP-like or VIP-like peptides refers to a peptide that has fewer amino acids than the PACAP-like or VIP-like peptide and has at least five contiguous amino acids (e.g. at least 10, 11, 12, 13, 14, 15, 16, 17, 18, 19, or 20 contiguous amino acids) with sequence similarity or identity (e.g., at least 90%, 95%, 97%, 99%, or 100% sequence identity over the at least 5-20 or more contiguous amino acids) to the PACAP-like or VIP-like peptide (e.g., one or more of the polypeptides of SEQ ID NOs: 1-70), respectively.

As used herein, the phrase “derivative” refers to a peptide that has been modified by the covalent attachment of another molecule and/or a functional group to the peptide chain. For example (but not by way of limitation), a derivative of a peptide may be produced by glycosylation, acetylation, pegylation, acylation, alkylation, oxidation, phosphorylation, sulfation, formylation, methylation, demethylation, amidation, gamma-carboxylation, cyclization, lactamization, prenylation, myristoylation, iodination, selenoylation, ribosylation, ubiquitination, or hydroxylation. The derivatized peptide can be a peptide analog. A derivative of a peptide can easily be made by standard techniques known to those of skilled in the art. A derivative of a peptide may possess an identical function(s) to the parent peptide. A derivative of a peptide may also have one or more other functions in addition to the function(s) of the parent peptide. For example (but not by way of limitation), a derivative of a peptide may have a longer half-life than the parent peptide and/or have cytoprotective or cytotoxic properties that are not possessed by the parent peptide.

As used herein, the phrase “subject” refers to a mammal, e.g., a non-primate (e.g., a cow, pig, horse, cat, dog, rat, etc.) or a primate (e.g., a monkey or a human being), most preferably a human being. In certain embodiments, the subject is a farm animal (e.g., a horse, pig, lamb or cow) or a pet (e.g., a dog, cat, rabbit, or monkey). In other embodiments, the subject is an animal other than a farm animal or a pet (e.g., a mouse, rat or guinea pig). In a preferred embodiment, the subject is a human. In another preferred embodiment, the subject is a human patient that has an untreated (but, e.g., diagnosed) or treated cancer.

As used herein, the phrase “in combination with” refers to the use of more than one therapeutic or cytoprotective agent in the methods of the invention. The use of the phrase “in combination with” does not restrict the order in which the therapeutic or cytoprotective agent is administered to a subject. One therapeutic or cytoprotective agent can be administered prior to, concomitantly with, or subsequent to the administration of a second or additional therapeutic or cytoprotective agent(s). The therapies are administered to a subject in a sequence and within a time interval such that the PACAP-like compound(s) of the present invention can act together with the other agent to provide a different response from the subject, preferably a greater therapeutic or cytoprotective benefit, than if they were administered otherwise.

As used herein, the phrase “nervous system” refers to the central nervous system (the brain and spinal cord), the sympathetic nervous system, the parasympathetic nervous system, and the enteric nervous system.

As used herein, the phrase “hematopoietic cell” refers to cells (including cancer cells) that are derived from hematopoietic stem cells. The normal cells of the body that are derived from hematopoietic stem cells include (but are not limited to) blood cells, e.g., erythrocytes, granulocytes (basophils, eosinophils and neutrophils), lymphocytes, monocytes (macrophages, microglia, splenocytes, and dendritic cells), and thrombocytes.

As used herein, the phrases “hematological malignancies” and “hematological cancers” refer to any cancer or malignancy of a hematopoietic cell, including (but not limited to) cancers of blood cells, cancers of nonstromal bone marrow cells, and cancers of the lymph node cells. These cancers include leukemias, lymphomas, and plasma cell dyscrasias.

As used herein, the phrase “plasma cell dyscrasias” refers to monoclonal neoplasms of the B-lymphocyte lineage, including (but not limited to) multiple myeloma, Waldenström's macroglobulinemia, POEMS syndrome, Seligmann's disease, and Franklin's disease.

By “treating,” “managing,” “reducing,” “inhibiting,” or “preventing” an injury to an organ of the body of a mammal, such as a human (e.g., the nervous system, the brain, the heart, the lung, the kidney, the liver, the kidneys, the pancreas, the gall bladder, the gastrointestinal tract (e.g., the pharynx, esophagus, stomach, small intestine (e.g., the duodenal mucosa), large intestine, appendix, and colon), the breast, the ovary, the testes, the prostate, the adrenal gland, the thymus, the spleen, or the lymph nodes) that results as an effect of treatment of the mammal with an anticancer agent is meant administering a PACAP-like compound of the invention to the mammal to ameliorate, alleviate, or hinder injury to one or more organs of the body of a mammal associated with, that results from, or that is likely to result from, treatment with an anticancer agent. By way of example only, administration of a PACAP-like compound provides treatment to a mammal by allowing an increase in the amount of an anticancer agent that can be administered to a mammal of at least about 1%, 2%, 5%, 8%, 10%, 15%, or 20% or more above the maximum tolerable dose normally administered to the mammal without an increase in, or with a diminishment of, organ injury (e.g., thereby providing a reduction or inhibition, or the prevention of, injury to an organ of the mammal). In another example of treatment, administration of a PACAP-like compound in combination with an anticancer agent reduces proliferation of a cancer cell by at least about 1%, 2%, 5%, 8%, 10%, 15%, 20%, 25%, 30% or more, relative to the proliferation of a cancer cell in the absence of administration of the PACAP-like compound. In another example of treatment, administration of a PACAP-like compound in combination with an anticancer agent improves the cancer survival rate (e.g., the five-year survival rate) of the mammal by at least 1, 2, 5, 10, 15, 20, 40, 60, 80, or 100% or more, relative to a mammal that does not receive the PACAP-like compound. Desirably, the methods of the present invention result in a decrease of 20, 40, 60, 80, or 100% in the size of a tumor or number of cancerous cells as determined using standard methods. Desirably, at least 20, 40, 60, 80, 90, or 95% of the treated mammals exhibit no injury to an organ of the body, greater tolerance to an anticancer agent, or a reduction in the size of a tumor or the number of cancer cells.

### DETAILED DESCRIPTION OF THE INVENTION

The inventors of the present patent application have discovered that 1) PACAP-like compounds protect against cellular injury caused by anticancer agents and that 2) PACAP-like compounds exhibit anticancer effects (e.g., the promotion of apoptosis) in certain cancer cells, in particular hematopoietic or hematological cancer cells (e.g., cancer cells derived from erythrocytes, granulocytes, lymphocytes, monocytes, and thrombocytes). Accordingly, the present invention features methods for treating, managing, reducing, inhibiting, or preventing an injury to an organ of a subject (e.g., the brain, the heart, the lungs, the liver, the kidneys, the pancreas, the gall bladder, the gastrointestinal tract (e.g., the pharynx, esophagus, stomach, small intestine (e.g., the duodenal mucosa), large intestine, appendix, and colon), the breasts, the ovaries, the testes, the prostate, the adrenal glands, the thymus, the spleen, or the lymph nodes) that results from administration of an anticancer agent by administering a PACAP-like compound to the subject.

Administration of the PACAP-like compound increases the maximum dose of an anticancer agent that can be tolerated by a subject by targeting one or more organs having a dose-limiting toxicity for the anticancer agent and treating, reducing, or inhibiting injury to the organ(s). The PACAP-like compound can also be administered to increase the efficacy of an anticancer agent against a cancer, e.g., a hematopoietic or hematological cancer.

The PACAP-like compound can be administered intravenously or intraarterially to the subject or by other routes described herein. For those subjects having an epithelial cell cancer (e.g., a cancer of the breasts, the ovaries, the testes, or the prostate, or a non-small cell lung cancer), the PACAP-like compound may be administered directly to one or more organs of the body (e.g., the brain, the heart, the lungs, the liver, the kidneys, the pancreas, the gall bladder, the gastrointestinal tract (e.g., the pharynx, esophagus, stomach, small intestine (e.g., the duodenal mucosa), large intestine, appendix, and colon), the adrenal glands, the thymus, the spleen, or the lymph nodes) that exhibit dose-limiting toxicity due to injury resulting from administration of an anticancer agent. For those subjects having a hematopoietic or hematological cancer (including, but not limited to, leukemias, lymphomas and plasma cell dyscrasias (e.g., multiple myeloma)) the PACAP-like compound may be administered indirectly (e.g., intravenous administration or any of the other non-direct routes described herein) to one or more organs of the body that exhibit dose-limiting toxicity due to injury resulting from administration of an anticancer agent.

In particular, the inventors of the present patent application have discovered that damage to cultured human renal tubule epithelial cells caused by cisplatin can be dramatically reduced by native human PACAP38, native human PACAP27, and analogs, fragments and derivatives of PACAP38 or PACAP27 (including, but not limited to, one or more of the polypeptides set forth in SEQ ID NOs: 1-70). The inventors of the present patent application have also discovered that the nephrotoxicity caused by cisplatin in mice in vivo can be dramatically reduced by native human PACAP38, native human PACAP27, and analogs, fragments and derivatives of PACAP38 or PACAP27 (including, but not limited to, one or more of the polypeptides set forth in SEQ ID NOs: 1-70). In an embodiment, the methods of the invention feature the direct (e.g., delivery to the kidney) or indirect (e.g., intravenous delivery) administration of a PACAP-like compound to a subject treated with, or to be treated with, cisplatin.

The inventors of the present patent application have discovered that the nephrotoxicity caused by doxorubicin in cultured human renal tubule epithelial cells can be dramatically reduced by native human PACAP38, native human PACAP27, and analogs, fragments and derivatives of PACAP38 or PACAP27 (including, but not limited to, one or more of the polypeptides set forth in SEQ ID NOs: 1-70). In an embodiment, the methods of the invention feature the direct (e.g., delivery into the kidney) or indirect (e.g., intravenous delivery) administration of a PACAP-like compound to a subject treated with, or to be treated with, doxorubicin.

The inventors of the present patent application have discovered that the pulmonary toxicity caused by bleomycin in cultured human lung cells can be dramatically reduced by native human PACAP38, native human PACAP27, and analogs, fragments and derivatives of PACAP38 or PACAP27 (including, but not limited to, one or more of the polypeptides set forth in SEQ ID NOs: 1-70). In an embodiment, the methods of the invention feature the direct (e.g., inhalation into the lungs) or indirect (e.g., intravenous delivery) administration of a PACAP-like compound to a subject treated with, or to be treated with, bleomycin.

The inventors of the present patent application have discovered that the toxicity caused by cisplatin in cultured rat pheochromocytoma cells can be reduced by native human PACAP38, native human PACAP27, and analogs, fragments and derivatives of PACAP38 or PACAP27. In an embodiment, the methods of the invention feature the direct administration of a PACAP-like compound to an organ of the body of a subject that is dose-limiting for an anticancer agent when that subject is treated for non-hematopoietic or non-hematological cancers (e.g., cancers of neuroendocrine origin) using an anticancer agent.

The inventors of the present patent application have discovered that the toxicity caused by doxorubicin in cultured human breast cancer cells can be reduced by native human PACAP38, native human PACAP27, and analogs, fragments and derivatives of PACAP38 or PACAP27. In an embodiment, the methods of the invention feature the direct administration of a PACAP-like compound to an organ of the body of a subject that is dose-limiting for an anticancer agent when that subject is treated for breast cancer using an anticancer agent.

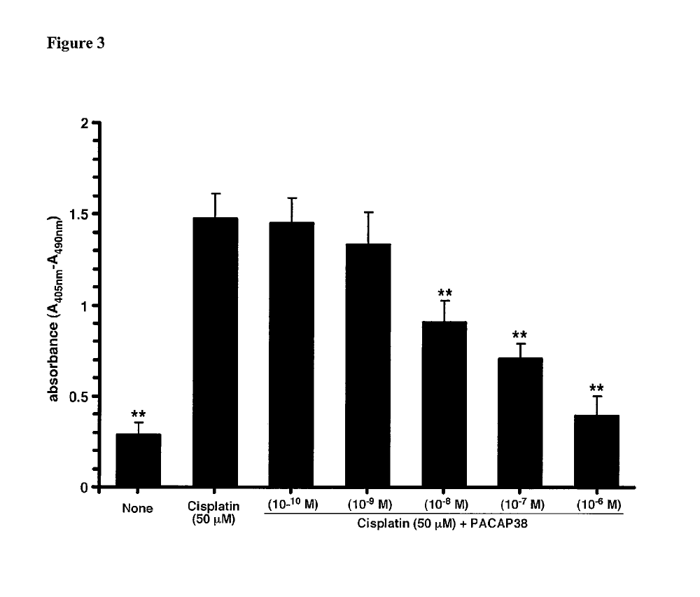

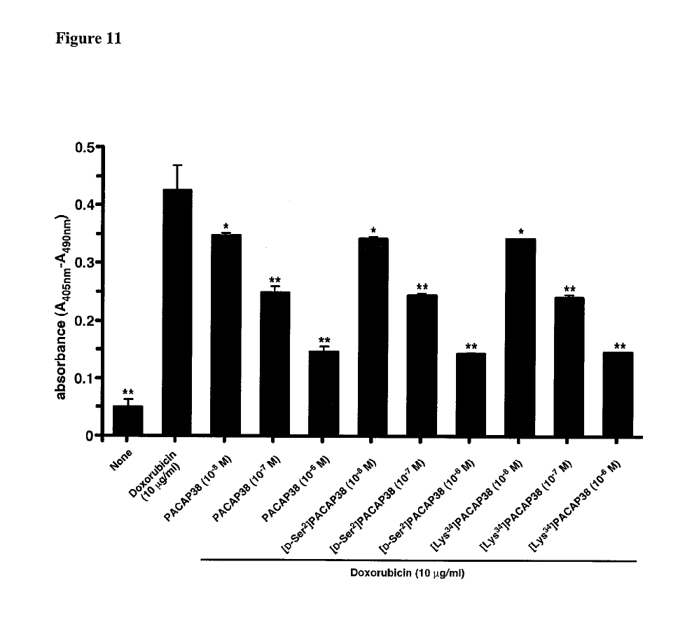

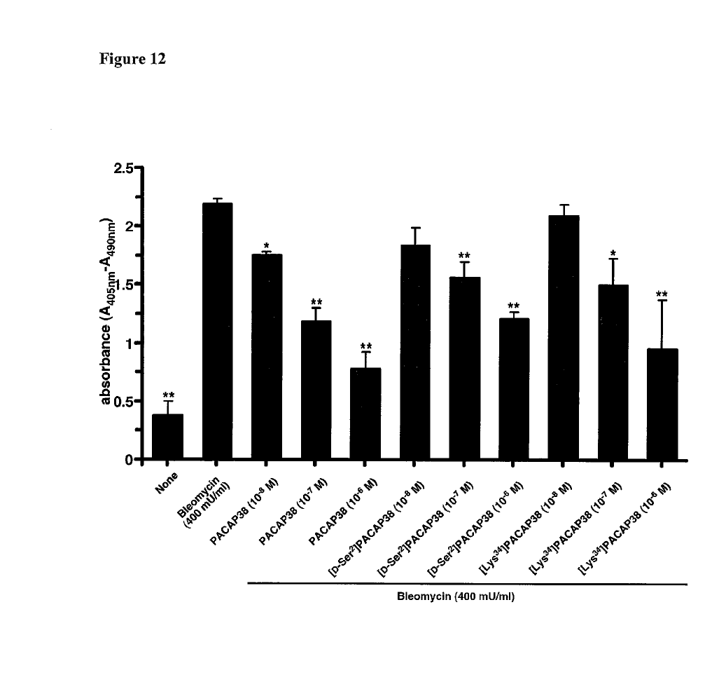

The inventors of the present patent application have discovered that the toxicity caused by etoposide in cultured human T-lymphocyte leukemia cells can only be slightly reduced by very high concentrations of native human PACAP38, and perhaps by even higher concentrations of native human PACAP27 or native human VIP. Therefore, PACAP38 is about 100-fold more potent as a cytoprotectant of kidney epithelial cells against toxicity due to treatment with cisplatin (FIG. 3) or doxorubicin (FIG. 11) and as a cytoprotectant of lung epithelial cells against toxicity caused by bleomycin (FIG. 12) than as a protectant of T-lymphocyte leukemia cells against toxicity caused by etoposide. In an embodiment, the methods of the invention feature the direct administration (e.g., administration directly to, e.g., the central nervous system or the lung) of a PACAP-like compound to a subject treated with etoposide for leukemia, although indirect administration (e.g., intravenous delivery) is not necessarily contraindicated in leukemic patients.

The inventors of the present patent application have discovered that the toxicity caused by doxorubicin in cultured human light-chain immunoglobulin-secreting myeloma cells can be directly enhanced by native human PACAP38, native human PACAP27, and analogs, fragments and derivatives of PACAP38 or PACAP27 (including, but not limited to, one or more of the polypeptides set forth in SEQ ID NOs: 1-70). In an embodiment, the methods of the invention feature the direct or indirect administration of a PACAP-like compound to a subject treated with, or to be treated with, e.g., doxorubicin.

The inventors of the present patent application have discovered that the toxicity caused by carmustine in cultured human light-chain immunoglobulin-secreting myeloma cells can be directly enhanced by native human PACAP38, native human PACAP27, and analogs, fragments and derivatives of PACAP38 or PACAP27 (including, but not limited to, one or more of the polypeptides set forth in SEQ ID NOs: 1-70). In an embodiment, the methods of the invention feature the direct or indirect administration of a PACAP-like compound to a subject treated with, or to be treated with, e.g., carmustine.

The inventors of the present patent application have discovered that the toxicity caused by vincristine in cultured human light-chain immunoglobulin-secreting myeloma cells can be directly enhanced by native human PACAP38, native human PACAP27, and analogs, fragments and derivatives of PACAP38 or PACAP27 (including, but not limited to, one or more of the polypeptides set forth in SEQ ID NOs: 1-70). In an embodiment, the methods of the invention feature the direct or indirect administration of a PACAP-like compound to a subject treated with, or to be treated with, e.g., vincristine.

The inventors of the present patent application have discovered that the toxicity caused by thalidomide in cultured human light-chain immunoglobulin-secreting myeloma cells can be directly enhanced by native human PACAP38, native human PACAP27, and analogs, fragments and derivatives of PACAP38 or PACAP27 (including, but not limited to, one or more of the polypeptides set forth in SEQ ID NOs: 1-70). The inventors of the present patent application have also discovered that the toxicity caused by thalidomide in cultured human erythroleukemia cells can be directly enhanced by native human PACAP38, native human PACAP27, and analogs, fragments and derivatives of PACAP38 or PACAP27 (including, but not limited to, one or more of the polypeptides set forth in SEQ ID NOs: 1-70). In an embodiment, the methods of the invention feature the direct or indirect administration of a PACAP-like compound to a subject treated with, or to be treated with, thalidomide.

The inventors of the present patent application have discovered that there is a correlation between whether PACAP-like compounds stimulate or inhibit the rate of proliferation of some cancer cells and whether PACAP-like compounds reduce or enhance the anticancer activity of some chemotherapeutics on some cancer cells. In particular, the inventors have discovered that PACAP-like compounds inhibit the rate of proliferation, or do not promote the proliferation, of most cancers of non-epithelial origin (e.g., hematopoietic or hematological cancers).

### Identification of PACAP-Like Compounds

The present invention provides methods for assaying and screening for PACAP-like compounds, such as PACAP38, PACAP27, VIP, their agonists, analogs, fragments, or derivatives, suitable for use in the method of the present invention by incubating the compounds with epithelial cells containing one or more PACAP/VIP receptors, e.g., kidney, liver, or lung epithelial cells, and then assaying for a reduction in a pathology-causing cell phenotype, e.g., in the presence of a chemotherapeutic agent. Alternatively, or in addition to the previous assay, PACAP-like compounds for use in the present invention can be identified by incubating the compounds with blood-derived cancer cells, e.g., multiple myeloma cells, and then assaying for the reduction or inhibition of cancer cell proliferation (Li et al., 2008).

For example, a PACAP-like compound that would be useful for the method of the present invention would exhibit the ability to promote an increase in the viability of cisplatin-treated kidney epithelial cells (e.g., by at least 2%, 5%, 10%, 20%, 25%, 30%, or more, relative to kidney epithelial cells not treated with the PACAP-like compound) and would promote a decrease in the rate of proliferation of multiple myeloma cells (e.g., by at least 2%, 5%, 10%, 20%, 25%, 30%, or more, relative to multiple myeloma cells not treated with the PACAP-like compound). In addition, the intrinsic activity of any PACAP-like compound at each of the three PACAP/VIP receptors can be determined in, e.g., stably transfected cell lines that express only one of these receptors by measuring the intracellular accumulation of cyclic AMP (Tatsuno et al., 2001). Radioligand receptor binding assays can be used to determine the affinity of a compound for one or more of the PACAP/VIP receptors. Such a compound may exhibit binding to a PACAP/VIP receptor with a dissociation constant of less than 10⁻⁶M, more preferably less than 10⁻⁷M, 10⁻⁸M, 10⁻⁹M, 10⁻10M, 10⁻¹¹M, or 10⁻¹²M, and most preferably less than 10⁻¹³M, 10⁻¹⁴M, or 10⁻¹⁵M. However, radioligand receptor binding assays do not differentiate between receptor agonists and receptor antagonists; other assays known in the art can differentiate PACAP/VIP receptor agonists from PACAP/VIP receptor antagonists.

The viability of renal, pulmonary and hepatic epithelial cells can be determined by a variety of techniques well known to those skilled in the art, including (but not limited to) quantification of the fragmentation of nuclear DNA, intracellular caspase 3 activity or extracellular lactate dehydrogenase activity, and counting of apoptotic (pyknotic) cells or Trypan blue-positive cells. In the preferred embodiment, the fragmentation of nuclear DNA or caspase 3 activity is determined.

The cell proliferation of multiple myeloma cells can be determined by a variety of techniques well known to those skilled in the art, including (but not limited to) quantification of the incorporation of bromodeoxyuridine or [³H]thymidine into nuclear DNA, counting of the number of cells expressing proliferating cell nuclear antigen and counting of mitotic figures. In the preferred embodiment, the incorporation of bromodeoxyuridine or [³H]thymidine into nuclear DNA is determined.

The intracellular accumulation of cyclic AMP in stably transfected cell lines that express only one of these receptors can be determined following stimulation with PACAP-like compounds by a variety of techniques well known to those skilled in the art, including (but not limited to) a radioimmunoassay or an enzyme-linked immunosorbent assay. The stimulation is stopped by the addition of ice-cold 20% trifluoroacetic acid. The cAMP is extracted from the cells, the extracts are centrifuged, the supernatants are placed into small plastic vials, and the supernatants are lyophilized for assay of the levels of cAMP. In the preferred embodiment, the intracellular levels of cAMP are quantified with an enzyme-linked immunosorbent assay.

### Patient Population

The present invention provides methods for treating, preventing, reducing, inhibiting, and managing damage or injury to one or more organs of the body, especially, the nervous system (e.g., the brain), heart, lung, kidneys, pancreas, gall bladder, gastrointestinal tract (e.g., the pharynx, esophagus, stomach, small intestine (e.g., the duodenal mucosa), large intestine, appendix, and colon), liver, adrenal gland, thymus, spleen, and lymph nodes, of subjects (e.g., humans or other mammals) treated with one or more anticancer agents. The methods involve the therapeutic or prophylactic administration of effective amounts of one or more compositions of the present invention.

The methods of the present invention involve the administration of one or more compositions of the invention (e.g., a composition that includes a PACAP-like compound, such as one or more of the PACAP-like polypeptides set forth in SEQ ID NOs: 1-70) to patients with one or more types of cancer who have suffered from, are suffering from, or are expected to suffer from the side-effects of one or more anticancer agents. In a preferred embodiment, the patient has been, is being, or is expected to be administered one or more cancer chemotherapeutics for the treatment of a hematological malignancy, including (but not limited to) a leukemia (e.g., chronic myelogenous leukemia or erythroleukemia), a lymphoma (e.g., Burkitt's lymphoma or mantle cell lymphoma), or a plasma cell dyscrasia (e.g., multiple myeloma or Waldenström's macroglobulinemia). These subjects may receive the composition by any of a number of routes of administration.

Alternatively, the patient population includes those patients having, who have suffered from, are suffering from, or are expected to suffer from a solid tumor of epithelial origin. These patients may be treated by targeting the compositions of the invention directly to the organ or tissue of the patient injured (or expected to be injured) by one or more of the anticancer agents administered to the patient (e.g., those organs that are dose-limiting for treatment with an anticancer agent). In an embodiment, the compositions of the invention are administered to these patients by a route other than parenteral administration.

The subjects may or may not have previously been treated on one or more occasions for one or more cancers. The subjects may or may not have previously been refractory to one or more cancer chemotherapeutics. The methods and compositions of the present invention may be used as an adjunctive treatment with a first line, second line or nonstandard treatment regimen for one or more cancers, especially hematopoietic or hematological cancer. The methods and compositions of the present invention may also be used as a substitute for the glucocorticoid in one of the standard glucocorticoid-containing regimens, including (but not limited to) CHOP, COP, COPP, MOPP, and VAD, for one or more cancers, especially a hematopoietic or hematological cancer. The methods and compositions of the present invention can be used before any side-effects of one or more cancer chemotherapeutics are observed or after the first or later observations of any side-effects of one or more cancer chemotherapeutics.

### Combination Therapies

The present invention also provides methods for treating, managing, reducing, inhibiting, or preventing injuries to one or more of the organs of the body of a humans or other mammal (e.g., injuries caused by or likely to result from administration of, e.g., one or more anticancer agents) by administering one or more compositions of the present invention in combination with one or more other cytoprotective agents. These other cytoprotective agents include (but are not limited to) amifostine, dexrazoxane, mesna, palifermin, and N-acetylcysteine.

Amifostine is an organothiophosphate prodrug that has been approved by the U.S. Food and Drug Administration (FDA) for the reduction of nephrotoxicity caused by repeated administration of cisplatin in patients with advanced ovarian cancer and for the reduction of xerostomia caused by radiation therapy in patients with head and neck cancer. Amifostine is dephosphorylated by alkaline phosphatase in the endothelium to yield the bioactive free thiol compound, which can scavenge free radicals. The administration of amifostine results in a significant but small protection of kidney function in cisplatin-treated patients, but causes serious side effects. A meta-analysis of five randomized clinical trials found that there were not sufficient data to determine whether amifostine can reduce the neuropathy caused by organoplatinum anticancer agents (Albers et al., 2007). Amifostine has even been reported to exacerbate bleomycin-induced lung injury (Ortiz et al., 1999).

Dexrazoxane is an EDTA-like compound that has been approved by the U.S. FDA for the reduction of the incidence and severity of cardiotoxicity caused by treatment with doxorubicin in women with advanced breast cancer. The chelation of free iron in the heart is believed to be responsible for its cardioprotective properties. A meta-analysis of nine randomized clinical trials confirmed the significant cardioprotective effect of treatment with dexrazoxane, but did not find any evidence for a difference in either response rate to anthracycline anticancer agents or in the length of survival (van Dalen et al., Cochrane Database Syst. Rev. (1):CD003917, 2005).

Mesna is a thiol-containing compound that has been approved by the U.S. FDA for the prevention of hemorrhagic cystitis caused by treatment with cyclophosphamide.

None of the listed cytoprotective agents stimulate G-protein-coupled receptors and all of these cytoprotective agents have mechanisms of action that are distinct from the presumed cytoprotective mechanisms of action of PACAP-like peptides. Therefore, one or more of these cytoprotective agents may exhibit additive or even synergistic effects when administered in combination with PACAP-like peptides.

### Synthesis of PACAP38, PACAP27, VIP, and Related Analogs

Except for a few unusual instances where incompatible chemistries are encountered, all analogs are prepared by modified Merrifield solid-phase procedures using Boc chemistries and hydrogen fluoride (HF) resin cleavage. Briefly, a Me-benzhydrylamine resin is used to yield amides directly after HF cleavage. Forty percent trifluoroacetic acid (TFA)/methylene chloride is used for Boc removal and couplings are achieved by diisopropylcarbodiimide (DIC) or TBTU/DIPEA activation or DIC/HOBt preactivation and active ester coupling. We estimate that approximately 20% of the couplings, which are monitored at each stage by the Kaiser ninhydrin test, fail to reach completion in 1 hour. Almost all of these resistant couplings can be driven to completion in 15-30 minutes by repeated coupling of the corresponding HOBt activated ester in dimethylformamide to which a catalytic amount of dimethylaminopyridine can be added for additional coupling power. CS Bio automated peptide synthesizers allow all of these pre-activations, double couplings, etc. to be fully automated with a concomitant increase in the speed of synthesis. Side-chain protection groups commonly used are: Asp and Glu, cHex; Ser and Thr, Bzl; Arg and His, tosyl (or Bom for His); Lys, 2-C1-Z; and Tyr, 2-Br-Z.

Peptides are simultaneously deprotected and cleaved from the resin support by treatment at 0° C. for 45 minutes with anhydrous HF containing 15% anisole. Excess HF is removed rapidly (˜10 minutes) under a rapid flow of dry nitrogen. With linear peptides, the resin is extracted with 2 M acetic acid and applied directly to preparative chromatography systems (either 1.5 or 2.5×25 cm columns) containing Vydac C-18 or phenyl-silica of 300-angstrom pore size (particle size 10 μm). Two fully volatile solvent elution systems have been used successfully for all of these peptides: linear gradient of acetonitrile in 0.1% TFA and acetonitrile in 20% acetic acid (excellent for insoluble peptides) at flow rates of about 8-20 ml/min. Gradients are generated with Rainin programmable high-performance liquid chromatography (HPLC) pumps and a typical separation run would normally be completed within 1 hour.

A long-chain saturated fatty acid is covalently linked to the free epsilon-amino group of one of the four Lys residues near the C-terminus of PACAP38 or one of the PACAP38 analogs (e.g., SEQ ID NO:5 and SEQ ID NO:6). PACAP27 and PACAP38 have similar affinities for the PAC₁, VPAC₁ and VPAC₂ receptors suggesting that the additional 11 amino acids are not essential for high-affinity receptor binding. The fatty acid attachment will promote high-affinity binding to serum albumin (Kurtzhals P. et al. J. Pharm. Sci. 85:304-308, 1996), which is by far the most abundant protein in the serum. This strategy has been used to make long-acting analogs of GLP-1 (Knudsen, L. B. et al. J. Med. Chem. 43:1664-1669, 2000), which is a member of the secretin/VIP/PACAP family.

The purity of each purified compound was confirmed by analytical HPLC, and structure by amino acid analysis (post-hydrolysis, pre-HPLC column labeling with fluorescamine) and matrix-assisted laser desorption/ionization (MALDI) mass spectroscopy.

### Demonstration of the Therapeutic Usefulness

The protocols and compositions of the present invention can first be tested in vitro, and then in preclinical models in vivo, for the desired therapeutic or prophylactic activity, prior to use in humans. For example, in vitro assays that can be used to determine whether administration of a specific therapeutic protocol is indicated, include in vitro cell culture assays in which an appropriate cell line or a patient tissue sample is grown in culture, and exposed to or otherwise administered a protocol, and the effect of such protocol upon the tissue sample is observed. For example (but not by way of limitation), rescuing of sensory neurons, renal or pulmonary epithelial cells, hepatocytes, or cardiomyocytes; decreased NFκB activation; decreased survival or proliferation of B- or T-lymphocytes; or decreased production of TNF-α and IL-6. A demonstration of one or more of the aforementioned properties of the exposed cells indicates that the therapeutic agent is effective for treating the condition in the patient. Many assays standard in the art can be used to assess such survival and/or growth of neurons, epithelial cells, hepatocytes, and/or B- or T-lymphocytes. Furthermore, any of the assays known to those skilled in the art can be used to evaluate the prophylactic and/or therapeutic utility of the combination therapies disclosed herein for treatment, management or prevention of injuries to one or more organs of the body caused by anticancer agents.

The injuries to one or more organs of the body of a subject caused by one or more anticancer agents can be monitored in the subject with commonly used biomarkers. For example (but not by way of limitation), injury to the kidney can be monitored by determining the concentration of protein in the urine, or the concentration of creatinine or urea nitrogen in the bloodstream. Injury to the liver can be monitored by determining the enzyme activity or concentration of alanine aminotransferase in the bloodstream, or the concentration of conjugated bilirubin in the urine. Injury to the heart can be monitored by determining the concentration of troponin I or the MB isoenzyme of creatinine kinase in the bloodstream. Injury to the β-cells of the pancreas can be monitored by determining the activity or concentration of glutamic acid decarboxylase in the bloodstream, and injury to the nervous system can be monitored by determining the activity or concentration of neuron-specific enolase in the bloodstream.

The injuries to one or more organs of the body of a subject caused by one or more anticancer agents can also be monitored in the subject with commonly used imaging techniques. For example (but not by way of limitation), injury to the heart can be monitored by electrocardiography or serial echocardiography.

The injuries to one or more organs of the body of a subject caused by one or more anticancer agents can also be monitored in the subject with commonly used functional tests. For example (but not by way of limitation), injury to the kidney can be monitored by determining the glomerular filtration rate with cystatin C or with sodium ¹²⁵I-iothalamate clearance. Injury to the peripheral nerves can be monitored by determining nerve conduction velocities or somatosensory perception. Injury to the heart can be monitored with a variety of exercise tests.

Based on the currently available data, there is a correlation between the reduction in the rate of proliferation of some cancer cells by PACAP-like compounds and the enhancement of the therapeutic efficacy of anticancer agents by PACAP-like compounds. Cancer cells can be obtained from either biopsy samples or, preferably, from circulating blood mononuclear cells from humans and other mammals, cultured in multi-well plates, and the effect of PACAP-like peptides on their rate of proliferation can be quantified in order to determine whether the PACAP-like compounds will protect the cancer cells against cancer chemotherapeutics or enhance the efficacy of cancer chemotherapeutics. Alternatively, the cancer cells could be screened for the induction of proapoptotic and anti-apoptotic genes by PACAP-like compounds using commonly-used microarray technology. The goal of these screening procedures will be to identify cancers that should respond to the addition of PACAP-like compounds to the anticancer regimen with an enhanced reduction of tumor burden. However, the addition of PACAP-like compounds to the anticancer regimen could still be beneficial to humans or other mammals even if the PACAP-like compounds do not reduce tumor burden because the reduction of injuries to one or more of the major organs of the body caused by the anticancer agents could still result in an increased length of survival and/or an increased quality of life. Alternatively, the addition of PACAP-like compounds to the anticancer regimen could also still be beneficial to humans or other mammals even if the PACAP-like compounds do not reduce tumor burden at the original doses of the anticancer agents. The reduction of injuries to one or more organs of the body caused by the anticancer agents should permit the use of higher doses of the anticancer agents, which should result in an enhanced reduction in tumor burden without an increase in the severity of the original side-effects.

The definitive diagnosis of multiple myeloma can be made in about 95% of the patients after a bone marrow aspiration or bone marrow biopsy. In the other patients, the bone marrow involvement is probably focal rather than diffuse. The efficacy of the adjunctive treatment with PACAP-like peptides can be determined subjectively by the patient reporting an improvement in symptoms, such as bone pain, fatigue, and overall well-being. The efficacy of the adjunctive treatment with PACAP-like peptides can be determined objectively by a physical examination that shows an improvement in overall appearance and muscle strength, by laboratory tests that show a reduction in anemia (a rise in hemoglobin and hematocrit), serum and urinary levels of the monoclonal paraprotein (Bence-Jones protein), and serum and urinary β-2 microglobulin, and by laboratory tests that show an improvement in kidney function (blood creatinine, urea nitrogen and cystatin C). In a preferred embodiment, serum and urinary levels of the monoclonal free light-chain immunoglobulin (Bence-Jones protein) are monitored with a highly sensitive nephelometric assay during the course of the treatment with the PACAP-like cytoprotective adjunctive agent. The definitive diagnosis of many leukemias can be made with the aide of biochemical genetic techniques, such as the polymerase chain reaction (PCR), or cytogenetic techniques, such as fluorescent in situ hybridization (FISH). For example, the diagnosis of chronic myelogenous leukemia can be made in circulating mononuclear cells with the aide of PCR for presence of the bcr-abl fusion gene or FISH for localization of the Philadelphia chromosome.

### Pharmaceutical Composition

The compositions of the present invention include bulk drug compositions useful in the manufacture of pharmaceutical compositions (e.g., impure or non-sterile compositions) and parenteral pharmaceutical compositions (i.e., compositions that are suitable for administration to a subject or patient) that can be used in the preparation of unit dosage forms. Such compositions comprise a prophylactically or therapeutically effective amount of a prophylactic and/or therapeutic agent disclosed herein or a combination of those agents and a pharmaceutically acceptable carrier or excipient. Preferably, compositions of the present invention comprise a prophylactically or therapeutically effective amount of one or more PACAP-like compounds useful in the Method of the invention and a pharmaceutically acceptable carrier or excipient. In a further embodiment, the composition of the present invention further comprises an additional therapeutic as discussed above.

In a specific embodiment, the term “pharmaceutically acceptable” means approved by a regulatory agency of the Federal government or listed in the U.S. Pharmacopeia or other generally recognized pharmacopeia for use in animals, and particularly for use in humans. The term “carrier” refers to a diluent, adjuvant (e.g., Freund's adjuvant or, more preferably, MF59C.I adjuvant), excipient, or vehicle with which the therapeutic is administered. The pharmaceutical carriers can be sterile liquids, such as water and oils, including those of petroleum, animal, vegetable or synthetic origin, such as peanut oil, soybean oil, mineral oil, sesame oil. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include (but are not limited to) starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. The composition, if desired, can also contain minor amounts of wetting or emulsifying agents, or pH buffering agents. These compositions can take many forms, including (but not limited to) suspensions, emulsions, tablets, pills, capsules, powders, and sustained-release formulations.

Generally, the ingredients of the compositions of the present invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the present invention can be formulated as neutral or salt forms. Pharmaceutically acceptable salts include (but are not limited to) those formed with anions such as those derived from hydrochloric acid, phosphoric acid, acetic acid, oxalic acid, and tartaric acid, and those formed with cations such as those derived from sodium, potassium, ammonium, calcium, ferric hydroxides, isopropylamine, triethylamine, 2-ethylamino ethanol, histidine, and procaine.

As desired, additives such as a dissolution aid (e.g., sodium salicylate or sodium acetate), a buffer (e.g., sodium citrate or glycerin), an isotonizing agent (e.g., glucose or invert sugar), a stabilizer (e.g., human serum albumin or polyethylene glycol), a preservative (e.g., benzyl alcohol or phenol), or an analgesic (e.g., benzalkonium chloride or procaine hydrochloride) may be added.

There are many delivery methods known to those skilled in the art that can be used to administer the PACAP-like compound(s), or the PACAP-like compound(s) in combination with other cytoprotective agents, in order to treat, manage, reduce, inhibit, or prevent injuries to one or more of the organs of the body of humans or other mammals caused, or likely to be caused, by one or more anticancer agents. For example (but not by way of limitation), encapsulation in liposomes, microparticles or microcapsules, secretion from mammalian cells genetically engineered to synthesize one or more PACAP-like peptides, or synthesis by various recombinant viral vectors. The routes of administration of the PACAP-like compounds of the present invention include (but are not limited to), parenteral (e.g., intradermal, intramuscular, intraperitoneal, intravenous, and subcutaneous), vaginal, rectal, epidural, and mucosal (e.g., intranasal, inhaled, and oral routes). In a specific embodiment, prophylactic or therapeutic agents of the present invention are administered intramuscularly, intravenously, intraosseously, or subcutaneously. The prophylactic or therapeutic agents may be administered by any convenient route or regimen, for example by infusion or a bolus injection, by absorption through epithelial or mucocutaneous linings (e.g., oral mucosa, rectal, topical, including buccal and sublingual, and intestinal mucosa, etc.) and may be administered in combination with other biologically active agents. Administration can be systemic or local.

In a specific embodiment, it may be desirable to administer the prophylactic or therapeutic agents of the present invention locally to the area in need of treatment; this maybe achieved by, for example, but not by way of limitation, local infusion, by injection, or by means of an implant, said implant being of a porous, non-porous, or gelatinous material, including membranes, such as Silastic membranes, or fibers.

In another embodiment, the compositions of this invention can be delivered in a controlled release or sustained release manner. In one embodiment, a pump can be used to achieve controlled or sustained release. In another embodiment, polymeric materials can be used to achieve controlled release or sustained release. Suitable polymers for controlled release or sustained release formulations include (but are not limited to) poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a controlled release or a sustained release formulation is inert, free of leachable impurities, stable on storage, sterile, and biodegradable. In a specific embodiment, a controlled release, or a sustained release device or formulation can be placed in proximity of the prophylactic or therapeutic target, thus reducing the required amount of the PACAP-like compound to only a fraction of the systemic dose. Many other techniques known to one skilled in the art can be used to produce controlled release or sustained release formulations comprising one or more therapeutic agents of the present invention.

The compositions for administration of the PACAP-like compounds include (but are not limited to) those suitable for oral, rectal, nasal, topical (including buccal and sublingual), vaginal, or parenteral (including subcutaneous, transcutaneous, intramuscular, intravenous, and intradermal) administration. The formulations may conveniently be presented in unit dosage forms and may be prepared by any methods well known in the art of pharmacy. Thus, the PACAP-like compounds of the present invention and their physiologically acceptable salts and solvates may be formulated for administration by inhalation or insufflation (either through the mouth or the nose), or by oral, parenteral or mucosal (such as buccal, vaginal, rectal, and sublingual) routes. In a preferred embodiment, parenteral administration is used.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium dodecyl sulfate). The tablets may be coated by methods well known in the art. Liquid preparations for oral administration may take the form of, for example, solutions, syrups or suspensions, or they may be presented as a dry product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated to give controlled release or sustained release of the active compound.

Amounts of the prophylactic or therapeutic agents for use according to the present invention may depend on the type of cancer, the type of anticancer agent, the method of administration, the severity of the injury to the patient's organ(s), and the general state of the patient, but generally range from about 1 μg to about 1 gram of the PACAP-like compound per dose (e.g., 1 μg, 2 μg, 5 μg, 7 μg, 10 μg, 20 μg, 50 μg, 70 μg, 100 μg, 200 μg, 500 μg, 700 μg, 1 mg, 2 mg, 5 mg, 7 mg, 10 mg, 20 mg, 50 mg, 70 mg, 100 mg, 200 mg, 500 mg, 700 mg, or 1 gram per dose). A dose of the PACAP-like compound can be administered therapeutically to a patient one or more times per hour, day, week, month, or year (e.g., 2, 4, 5, 6, 7, 8, 9, 10, 11, or 12 times per hour, day, week, month, or year).

For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in a powder form for reconstitution before use with a suitable vehicle, e.g., sterile pyrogen-free water. The PACAP-like compounds of the present invention may be administered to the subject by intravenous infusion at a rate of between 1 pmol/kg body weight/hour to 1200 pmol/kg body weight/hour. The rate of intravenous infusion may also be between 1-200 pmol/kg body weight/hour or between 100-200 pmol/kg body weight/hour. The rate of intravenous infusion may also be between 200-600 pmol/kg body weight/hour. The intravenous infusion of the PACAP-like compound may be for between 1 to 12 hours or more (e.g., 24, 36, or 48 hours or more). Administration of the PACAP-like compound may be repeated one or more times over the course of an hour, a day, a week, a month, or a year (e.g., 2, 3, 4, 5, 6, 7, 8, 9, 10, 11, or 12 or more times).

In addition to the formulations described previously, the prophylactic or therapeutic agents may also be formulated as a depot preparation. Such long-acting formulations may be administered by implantation (e.g., subcutaneously or intramuscularly) or by intramuscular injection. Thus, for example, the prophylactic or therapeutic agents may be formulated with suitable polymeric or hydrophobic materials (e.g., as an emulsion in an acceptable oil) or ion-exchange resins, or as sparingly soluble derivatives, for example, as a sparingly soluble salt.

Compositions suitable for topical administration to the skin may be presented as ointments, creams, gels, and pastes comprising the compound and a pharmaceutically acceptable carrier. For example (but not by way of limitation), a suitable topical delivery system is a transdermal patch containing the PACAP-like compound to be administered.

Sublingual tablets can be prepared by using binders (e.g., hydroxypropylcellulose, hydroxypropylmethylcellulose, or polyethylene glycol), disintegrating agents (e.g., starch or carboxymethylcellulose calcium), and/or lubricants (e.g., magnesium stearate or talc).

Suitable formulations for nasal administration wherein the carrier is a solid include a coarse powder having a particle size, for example, in the range 20 to 500 microns (μm). Suitable formulations for nasal administration wherein the carrier is a liquid (e.g., a nasal spray or nasal drops) include aqueous or oily solutions of the active ingredient.

Compositions suitable for parenteral administration include aqueous and non-aqueous sterile injection solutions which may contain anti-oxidants, buffers, bacteriostatic agents, and solutes that make the formulation isotonic with the blood of the intended recipient; and aqueous and non-aqueous sterile suspensions that may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described. It should be understood that in addition to the ingredients specifically mentioned above, the formulations of this invention may include other agents commonly used in the art for the type of formulation in question. For example (but not by way of limitation), those suitable for oral administration may include flavoring agents.

### Gene Therapy

In a specific embodiment, a series of nucleic acids that encode for one or more PACAP-like peptides that are useful for the method of the present invention are administered alone or as part of a suitable vector in order to treat, manage or prevent injuries to one or more of the organs of the body of humans or other mammals caused by one or more anticancer agents. The series of nucleic acids are then translated in the body of the subject to produce one or more PACAP-like peptides that have a prophylactic or therapeutic effect. Many different gene therapy methods can be used to administer one or more of the PACAP-like peptides. Some gene therapy methods that can be used to administer the PACAP-like peptides of this invention in order to treat, manage or prevent injuries to one or more of the organs of the body of humans or other mammals caused by one or more anticancer agents are described below. These examples are only for illustrative purposes. Those skilled in the art of recombinant DNA technology will recognize that there are many other variants that can be used for the same purposes.

The nucleic acid polymers that code for the PACAP-like peptide(s) can be administered as naked DNA (as an expression vector), or preferably encapsulated in liposomes or microparticles. The nucleic acid polymers can contain a promoter sequence, preferably a heterologous promoter sequence, preceding the sequence that codes for the PACAP-like peptide(s). The heterologous promoter sequence can provide for either constitutive or inducible expression of the PACAP-like peptide(s). In addition, the promoter sequence can provide for cell type-specific expression. The liposomes or microparticles can also contain one or more targeting vectors, such as a bioactive peptide or a monoclonal antibody, in order to direct the whole complex preferentially to one or more types of cells.

The nucleic acid polymers that code for the PACAP-like peptide(s) can be administered after incorporation into a viral vector. The viral vectors that can be used to administer the PACAP-like peptides of this invention include (but are not limited to) adenovirus vectors, adeno-associated virus vectors, lentivirus vectors, herpesvirus vectors, and poxvirus vectors. The incorporated nucleic acid polymers in the viral vector can contain a promoter sequence, preferably a heterologous promoter sequence, preceding the sequence that codes for the PACAP-like peptide(s). The heterologous promoter sequence can provide for either constitutive or inducible (e.g., van de Loo, F. A. et al. Curr. Opin. Mol. Ther. 6:537-545, 2004) expression of the PACAP-like peptide(s). In addition, the promoter sequence can provide for cell type-specific expression (e.g., Wang, B. et al. Gene Ther 15:1489-1499, 2008). The viral vector can be pseudotyped or cross-packaged (e.g., Rabinowitz, J. E. et al. J. Virol. 76:791-801, 2002) in order to direct the viral vector preferentially to one or more types of cells.

The nucleic acid polymers that code for the PACAP-like peptide(s) can be administered after ex vivo transfection into mammalian cells. The mammalian cells, preferably the subject's own cells, that can used to administer the PACAP-like peptides of this invention include (but are not limited to) mesenchymal stem cells, hematopoietic stem cells, neural stem cells, liver stem cells, and various differentiated mammalian cells. Those skilled in the art of recombinant DNA technology will be familiar with numerous techniques for transfecting mammalian cells with nucleic acid polymers. The transfected nucleic acid polymers can either integrate into the host cell DNA or form a translation-competent episomal complex in the host cell nucleus. The incorporated nucleic acid polymers in the viral vector can contain a promoter sequence, preferably a heterologous promoter sequence, preceding the sequence that codes for the PACAP-like peptide(s). The heterologous promoter sequence can provide for either constitutive or inducible expression of the PACAP-like peptide(s). In addition, the promoter sequence can provide for cell type-specific expression.

### EXAMPLES

In order to make the uses of the present invention clearer, the following examples are presented. These examples are only for illustrative purposes and should not be interpreted in any way as limitations in the uses of this invention.

### Example 1

### Reduction of Cisplatin-Induced Cytotoxicity by PACAP and PACAP Analogs

Cisplatin (cis-diamminedichloridoplatinum(II), Platinol) is the first-in-class platinum-based DNA-crosslinking anticancer therapeutic. It was approved for clinical use by the U.S. FDA in 1978. The other members of this class of alkylating-like platinum-based anticancer agents now include (but are not limited to) carboplatin, oxaliplatin and satraplatin. Cisplatin is one of the most widely used cancer chemotherapeutics and is the cornerstone of many multi-drug anticancer regimens. Nephrotoxicity is usually the dose-limiting toxicity for the use of cisplatin in cancer chemotherapy, but sensory neuropathies can sometimes limit the doses that can be used to treat some patients.

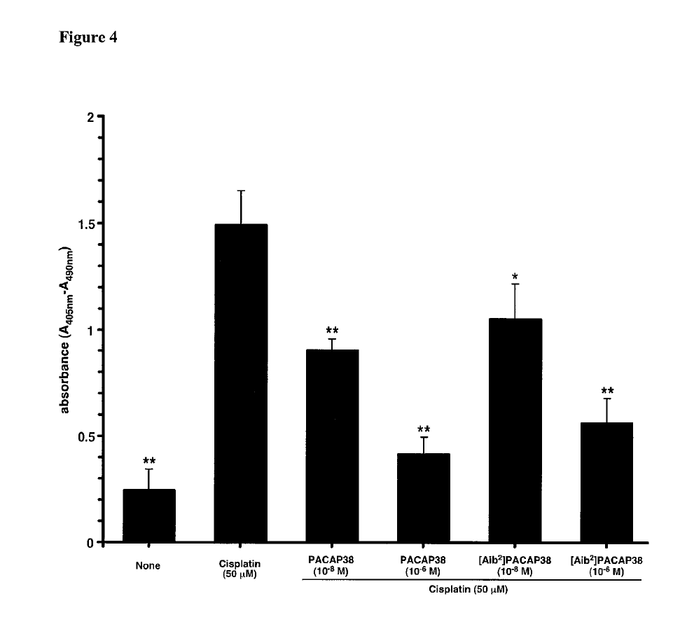

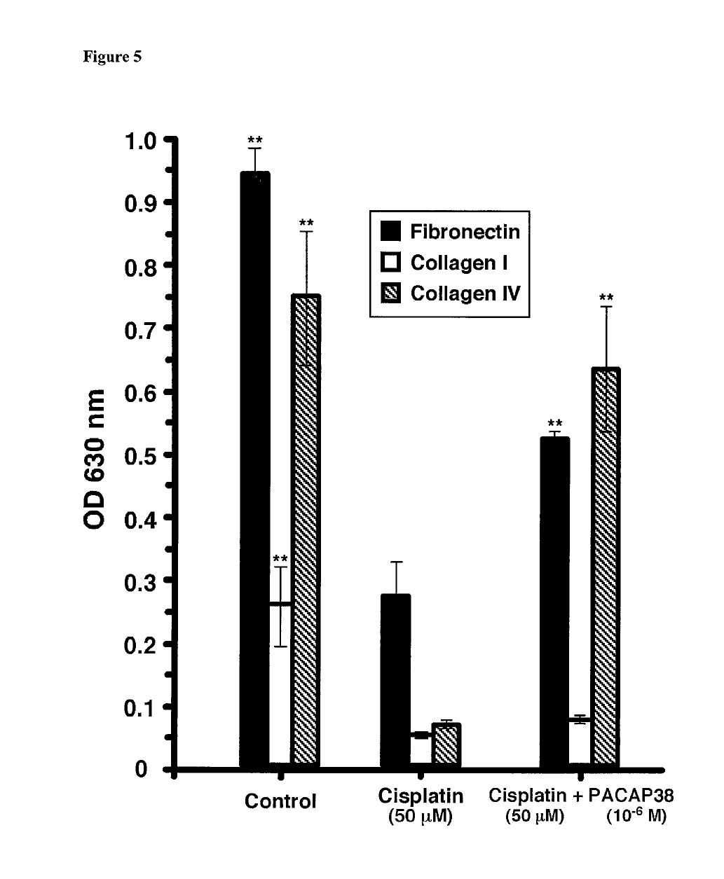

Treatment of human renal proximal tubule epithelial cells with cisplatin resulted in a large significant increase in apoptotic cell death (FIG. 3). The addition of PACAP38 to the medium resulted in a significant dose-dependent reduction in cisplatin-induced apoptotic cell death of the human renal proximal tubule epithelial cells. At the highest dose tested, PACAP38 almost completely prevented the apoptotic cell death caused by cisplatin. PACAP38 and the more proteolysis-resistant analog [Aib²]PACAP38 were similarly effective as protectants against cisplatin-induced apoptotic cell death of human renal proximal tubule epithelial cells (FIG. 4). Treatment of human renal proximal tubule epithelial cells with cisplatin also resulted in a large significant decrease in the attachment of these cells to either a fibronectin-based or a collagen IV-based matrix (FIG. 5). The addition of PACAP38 to the medium significantly reversed the inhibitory effect of cisplatin on the binding of the human renal proximal epithelial cells to either a fibronectin-based or a collagen IV-based matrix.

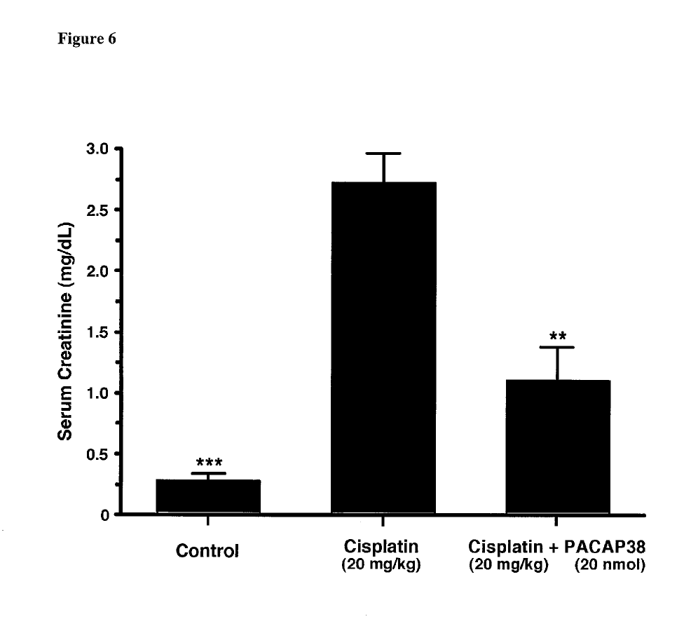

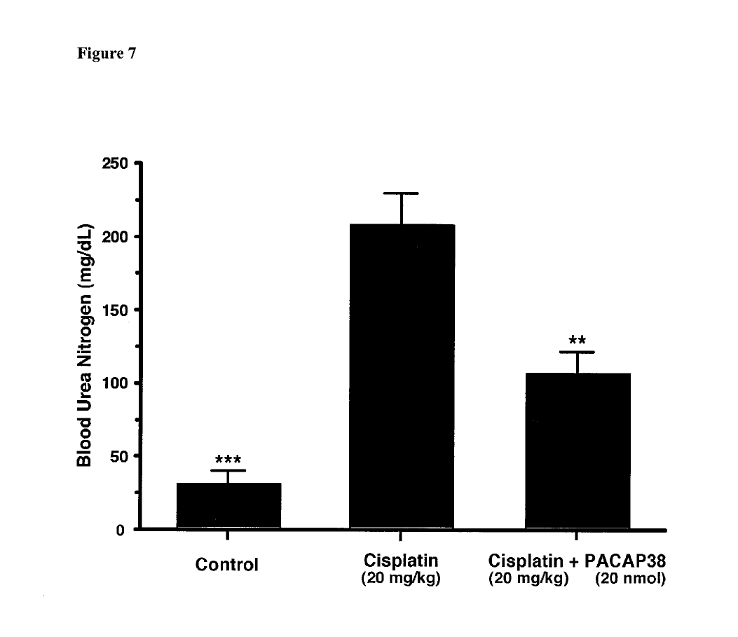

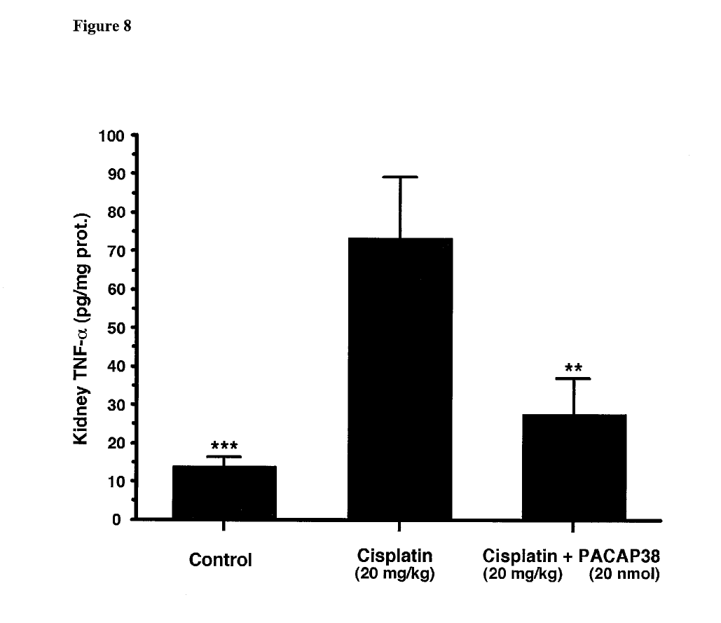

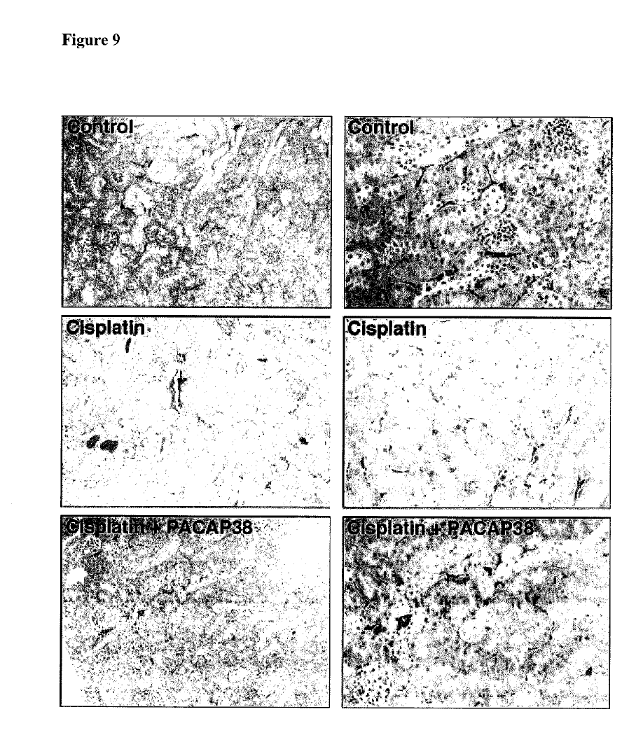

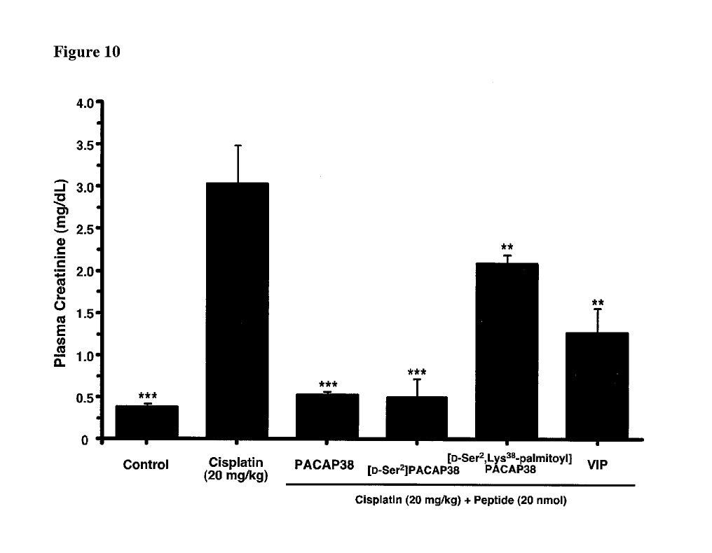

The cytoprotective effect of PACAP38 against cisplatin-induced nephrotoxicity was also seen in a common in vivo model. Male C57BL/6 mice were given a single intraperitoneal injection of 20 mg/kg of cisplatin. Twenty nanomoles of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. The mice treated with cisplatin had significantly increased levels of serum creatinine, blood urea nitrogen and TNF-α in the kidney compared to the saline-injected control group (FIGS. 6, 7 and 8). The kidneys from the cisplatin-injected mice showed extensive tubular damage, tubular dilations, intratubular debris, and intratubular casts (FIG. 9). Treatment of the cisplatin-injected mice with PACAP38 significantly reduced the increases in serum creatinine, blood urea nitrogen and TNF-α in the kidney (FIGS. 6, 7 and 8). The treatment with PACAP also decreased the extensive histological damage caused by cisplatin (FIG. 9). PACAP38 and [D-Ser²]PACAP38 were approximately equipotent as cytoprotectants against cisplatin-induced impairment of kidney function, while [D-Ser²,Lys³⁸-palmitoyl]PACAP38 and VIP were less potent as renoprotectants (FIG. 10).

These experiments show that PACAP38 is a potent cytoprotectant against cisplatin-induced damage to the kidney, which is the dose-limiting toxicity for cancer chemotherapy with cisplatin. Therefore, pre- and/or post-treatment of subjects undergoing cisplatin-based cancer chemotherapy with therapeutic doses of PACAP38 and/or PACAP analogs should result in a higher maximal tolerable dose of cisplatin, and an increased frequency of partial clinical responses and/or an increased number of complete remissions.

### Example 2

### Reduction of Doxorubicin-Induced Renal Toxicity by PACAP and PACAP Analogs

Daunorubicin, which is made by the actinobacterium Streptomyces peucetius, was the first anthracycline anticancer therapeutic to be discovered. Doxorubicin (14-hydroxydaunorubicin, Adriamycin) was developed shortly afterwards. The other anthracycline anticancer agents used clinically now include (but are not limited to) epirubicin, idarubicin and valrubicin. Doxorubicin is a potent inhibitor of both DNA and RNA synthesis and is one of the most widely used cancer chemotherapeutics. It is commonly used to treat leukemias, lymphomas, multiple myeloma, and cancers of the breast, bladder, uterus, ovaries, and lung. Cardiotoxicity is usually the dose-limiting toxicity for the use of doxorubicin in cancer chemotherapy, but nephrotoxicity can sometimes limit the doses that can be used to treat some patients.

Treatment of human renal proximal tubule epithelial cells with doxorubicin resulted in a large significant increase in apoptotic cell death (FIG. 11). The addition of PACAP38, [D-Ser²]PACAP38 or [Lys³⁴]PACAP38 to the medium resulted in a significant dose-dependent reduction in doxorubicin-induced apoptotic cell death of the human renal proximal tubule epithelial cells. At the highest dose tested (10⁻⁶ M), all three peptides reduced the apoptotic cell death of these epithelial cells by more than 60%. These experiments show that PACAP38 and PACAP analogs can protect the kidney against the toxic side-effects of multiple anticancer agents (FIGS. 3-11).

### Example 3

### Reduction of Bleomycin-Induced Pulmonary Toxicity by PACAP and PACAP Analogs

Bleomycin, which is a family of glycopeptides made by the actinobacterium Streptomyces verticillus, was discovered in 1962. Bleomycin (Blenoxane) was approved for clinical use by the U.S. FDA in 1973. It causes DNA strand breaks and is used to treat Hodgkin's lymphoma, squamous cell carcinomas and testicular cancer. Pulmonary toxicity is usually the dose-limiting toxicity for the use of bleomycin in cancer chemotherapy

Treatment of human pulmonary epithelial cells with bleomycin resulted in a large significant increase in apoptotic cell death (FIG. 12). The addition of PACAP38, [D-Ser²]PACAP38 or [Lys³⁴]PACAP38 to the medium resulted in a significant dose-dependent reduction in bleomycin-induced apoptotic cell death of the human pulmonary epithelial cells. At the highest dose tested (10⁻⁶ M), all three peptides reduced the apoptotic cell death of these epithelial cells by more than 60%. These experiments show that PACAP38 and PACAP analogs are potent cytoprotectants against bleomycin-induced damage to the lung, which is the dose-limiting toxicity for cancer chemotherapy with bleomycin. Therefore, pre- and/or post-treatment of subjects undergoing bleomycin-based cancer chemotherapy with therapeutic doses of PACAP38 and/or PACAP analogs should result in a higher maximal tolerable dose of bleomycin, and an increased frequency of partial clinical responses and/or an increased number of complete remissions.

### Example 4

### Reduction of Cisplatin-Induced Apoptotic Cell Death of Pheochromocytoma Cells by PACAP and PACAP Analogs

Pheochromocytomas are catecholamine-secreting tumors that are derived from the adrenal medulla and the sympathetic ganglia. About 10% of these tumors have an extramedullary origin The most critical symptom is hypertension, which can be fatal. Plasma levels of catecholamines, chromogranin A and dopamine-β-hydroxylase are usually elevated in patients with pheochromocytomas.

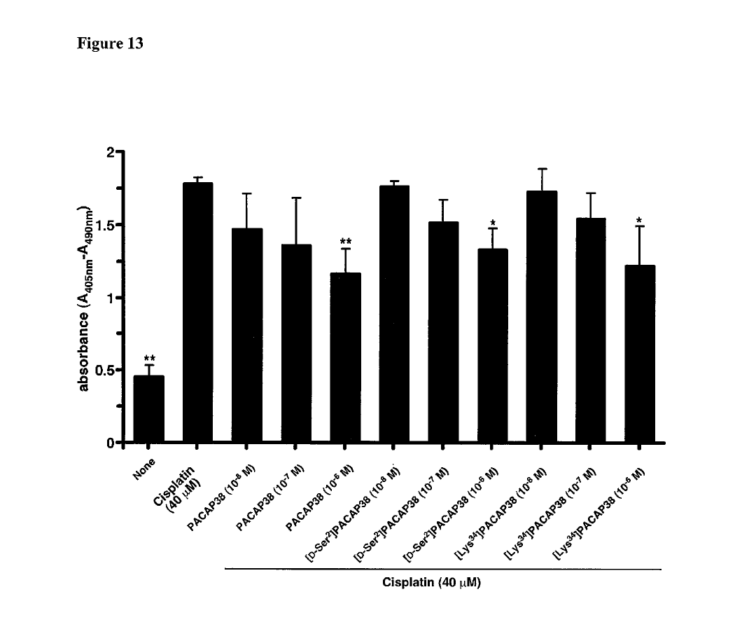

Treatment of rat pheochromocytoma cells with cisplatin resulted in a large significant increase in apoptotic cell death (FIG. 13). The addition of PACAP38, [D-Ser²]PACAP38 or [Lys³⁴]PACAP38 to the medium resulted in a dose-dependent reduction in cisplatin-induced apoptotic cell death of the neuroendocrine tumor cells. At the highest dose tested (10⁻⁶ M), all three peptides significantly reduced the apoptotic cell death of these pheochromocytoma cells. These experiments show that PACAP38 and PACAP analogs can protect neuroendocrine tumor cells against cisplatin-induced damage.

### Example 5

### Reduction of Doxorubicin-Induced Apoptotic Cell Death of Breast Cancer Cells by PACAP and PACAP Analogs

Breast cancer is the second most prevalent type of cancer in the world.

Breast cancer is more than 100-fold more prevalent in women than in men. Women in the USA have the highest incidence of breast cancer in the world. Doxorubicin is frequently used as a component of multidrug adjuvant regimens following local surgical or radiation therapy, or as a component of multidrug regimens for the treatment of metastatic breast cancer.

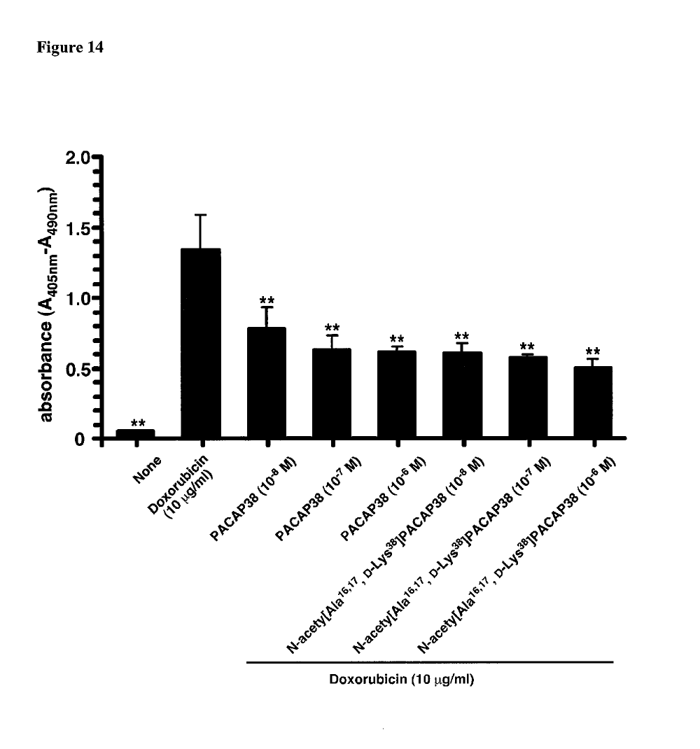

Treatment of human breast cancer cells with doxorubicin resulted in a large significant increase in apoptotic cell death (FIG. 14). The addition of PACAP38 or N-acetyl[Ala^{16,17},D-Lys³⁸]PACAP38 to the medium resulted in a dose-dependent reduction in doxorubicin-induced apoptotic cell death of the estrogen-dependent breast cancer cells. At the highest dose tested (10⁻⁶ M), both peptides reduced the apoptotic cell death of these breast cancer cells by more than 50%. N-acetyl[Ala^{16,17},D-Lys³⁸]PACAP38 seemed to be slightly more potent than PACAP38. These experiments show that PACAP38 and PACAP analogs can protect breast cancer cells against doxorubicin-induced damage.

### Example 6

### Reduction of Etoposide-Induced Apoptotic Cell Death of Leukemia Cells by PACAP27 and PACAP38

Etoposide is an inhibitor of topoisomerase II, an enzyme that is crucial for DNA replication and cell proliferation. Etoposide is derived from the plant toxin podophyllotoxin and is used to treat testicular cancer, lung cancer, lymphomas, non-lymphocytic leukemia, and glioblastoma multiforme. It is frequently used clinically as part of a multidrug regimen. The other members of this class of podophyllotoxin-derived anticancer agents include (but are not limited to) teniposide. Myelosuppression is usually the dose-limiting toxicity for the use of etoposide in cancer chemotherapy, but nephrotoxicity or bladder toxicity can sometimes limit the doses that can be used to treat some patients.

Leukemias are cancers of the white blood cells (leukocytes), including granulocytes, monocytes and lymphocytes. About 2% of the diagnosed cancers in the USA are leukemias.

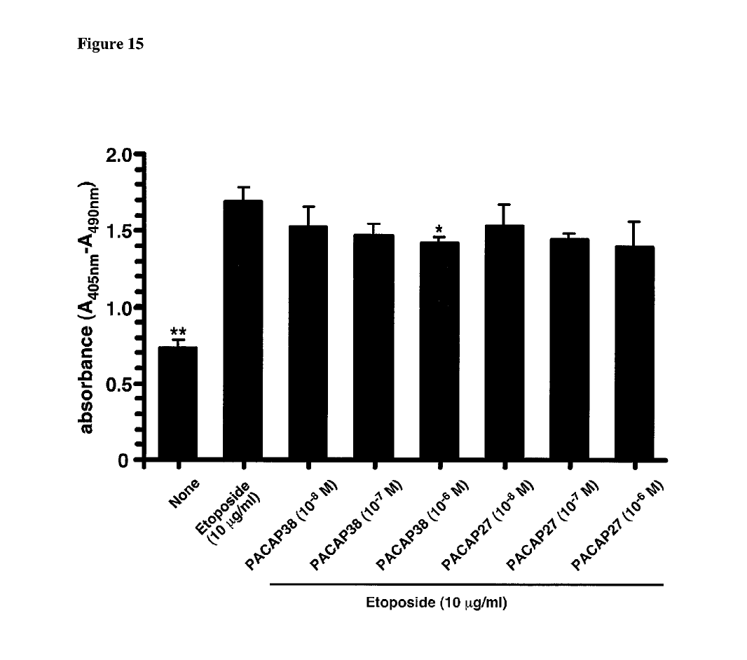

Treatment of human T-lymphocyte leukemia cells with etoposide resulted in a large significant increase in apoptotic cell death (FIG. 15). The addition of PACAP27 or PACAP38 to the medium resulted in a small reduction in etoposide-induced apoptotic cell death of the human leukemia cells. The reduction of the apoptotic cell death of these hematological cancer cells was only significant at the highest dose of PACAP38 tested (10⁻⁶ M), while PACAP27 did not have a significant effect at any of the doses tested. These experiments show that PACAP27 and PACAP38 have, at best, only a minimal protective effect against etoposide-induced apoptosis of these human T-lymphocyte leukemia cells.

### Example 7

### Enhancement of Doxorubicin-Induced Apoptotic Cell Death of Myeloma Cells by PACAP and PACAP Analogs

Multiple myeloma, a malignant cancer of plasma cells, is the sixth most common cancer in the USA. It accounts for about 10% of the hematological malignancies diagnosed in the USA. Multiple myeloma is slightly more prevalent in men than in women. The disease can cause serious medical complications, including bone resorption (osteolysis), hypercalcemia, anemia, thrombocytopenia, and kidney failure. Inflammation of the kidney is the second most frequent complication and occurs in about half of the patients with multiple myeloma. The cause of this inflammation is the overproduction by plasma cells of light-chain immunoglobulins (Bence-Jones proteins), which aggregate to form casts in the distal convoluted tubules and collecting ducts of the kidneys. Plasma cells are derived from activated B lymphocytes by clonal expansion. The normal restraints on the expansion of a single plasma cell clone is lost in patients with multiple myeloma, which results in the excessive production of a single type of light-chain immunoglobulin.

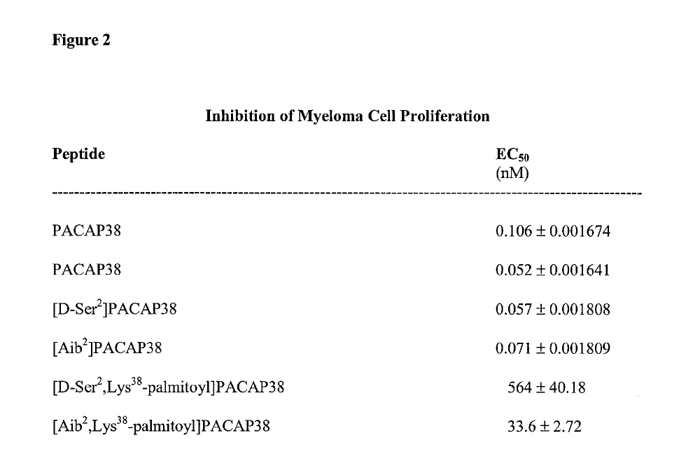

The effects of PACAP38 and PACAP38 analogs on myeloma cell proliferation were assessed by determining incorporation of bromodeoxyuridine into DNA during cell division. The number of myeloma cells approximately doubled during the 24-hour incubation period in the absence of treatment with PACAP-like peptides. The addition of PACAP38, [D-Ser²]PACAP38 or [Aib²]PACAP38 to the medium resulted in 50% inhibition of the rate of proliferation of the light-chain immunoglobulin-secreting human myeloma cells at concentrations lower than 100 picomolar (FIG. 2). PACAP38 can also directly protect the kidney against light-chain immunoglobulin overload and would be expected to inhibit bone resorption in patients with multiple myeloma (Li et al., 2008). Therefore, PACAP and PACAP analogs might be useful monotherapeutics for patients with multiple myeloma.

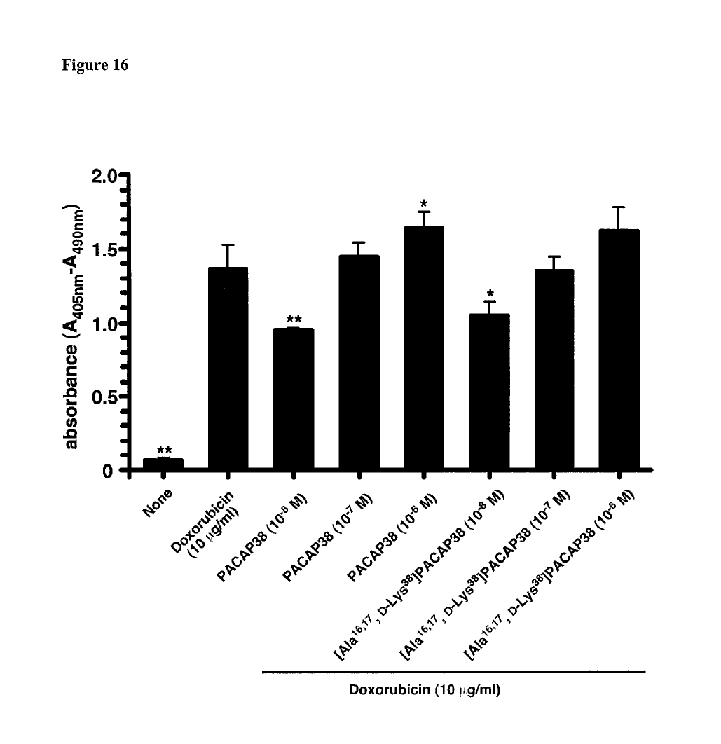

Treatment of human myeloma cells with doxorubicin resulted in a large significant increase in apoptotic cell death (FIG. 16). The addition of PACAP38 or [Ala^{16,17},D-Lys³⁸]PACAP38 to the medium resulted in a biphasic dose-response effect on doxorubicin-induced apoptotic cell death of the human myeloma cells. At the lowest dose tested (10⁻⁸ M), both peptides reduced the doxorubicin-induced apoptotic cell death of these malignant plasma cells. However, at the highest dose tested (10⁻⁶ M), PACAP38 significantly enhanced the doxorubicin-induced apoptotic cell death of these malignant plasma cells, while [Ala^{16,17},D-Lys³⁸]PACAP38 produced a small, but not significant, enhancement of the doxorubicin-induced apoptosis.

### Example 8

### Enhancement of Carmustine-Induced Apoptotic Cell Death of Myeloma Cells by PACAP and PACAP Analogs

Carmustine (α-chloro-nitrosourea) is a mustard gas-related alkylating anticancer therapeutic that is used to treat gliomas, medulloblastomas, astrocytomas, multiple myeloma, and Hodgkin's lymphoma and non-Hodgkin lymphomas. The other members of this class of nitrosourea anticancer agents include (but are not limited to) lomustine, semustine and streptozotocin. Myelosuppression is usually the dose-limiting toxicity for the use of carmustine in cancer chemotherapy, but hepatotoxicity or pulmonary toxicity can sometimes limit the doses that can be used to treat some patients.

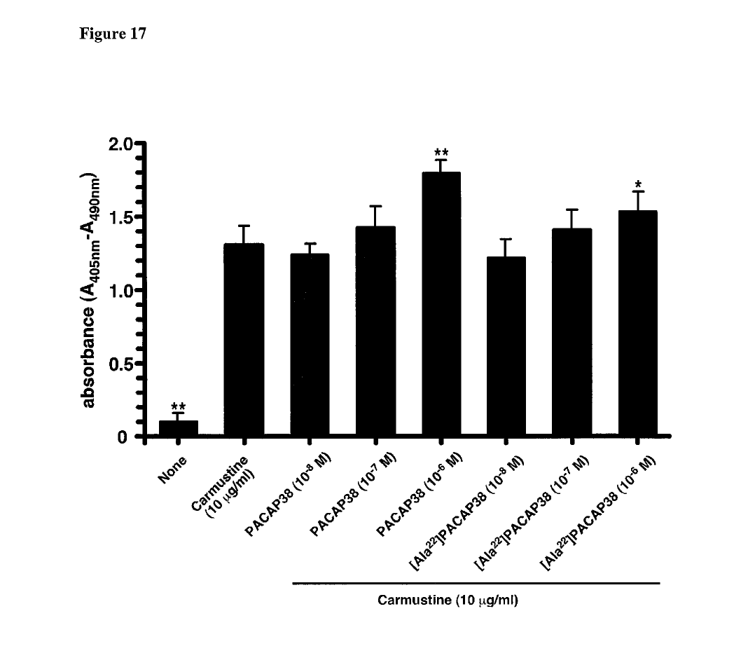

Treatment of human myeloma cells with carmustine resulted in a large significant increase in apoptotic cell death (FIG. 17). The addition of PACAP38 or [Ala²²]PACAP38 to the medium resulted in an enhancement of carmustine-induced apoptotic cell death of the human myeloma cells. At the highest dose tested (10⁻⁶ M), both peptides significantly enhanced the carmustine-induced apoptotic cell death of these malignant plasma cells. PACAP38 appeared to be more potent than [Ala²²]PACAP38 as an enhancer of carmustine-induced apoptotic cell death of the human myeloma cells.

### Example 9

### Enhancement of Vincristine-Induced and Thalidomide-Induced Apoptotic Cell Death of Myeloma Cells by PACAP38

Vincristine (Oncovin) is a vinca alkaloid anticancer therapeutic that is used to treat many different cancers, including (but are not limited to) Hodgkin's and non-Hodgkin lymphomas, leukemias, breast cancer, neuroblastoma, and multiple myeloma. It was approved for use as an anticancer agent by the U.S. FDA in 1963. The other members of this class of vinca alkaloid anticancer agents include (but are not limited to) vinblastine and vinorelbine. Peripheral neuropathy is usually the dose-limiting toxicity for the use of vincristine in cancer chemotherapy, but bone marrow suppression or nausea can sometimes limit the doses that can be used to treat some patients. Thalidomide was originally marketed in the late 1950s and early 1960s as a sedative/hypnotic, and was removed from use because it caused severe birth defects. Thalidomide was later developed as a treatment for leprosy and was approved by the U.S. FDA in 1998 for the treatment of erythema nodosum leprosum. In 2006, the U.S. FDA approved thalidomide (Thalomid) in combination with dexamethasone for the treatment of multiple myeloma. The other members of this class of anticancer agents include (but are not limited to) lenalidomide (CC-5013, Revlimid), which was approved by the U.S. FDA in 2005 for the treatment of multiple myeloma, and CC-4047 (Actimid), which is in clinical trials. Peripheral neuropathy is usually the dose-limiting toxicity for the use of thalidomide in cancer chemotherapy, but deep vein thrombosis, nephrotoxicity, or hypotension can sometimes limit the doses that can be used to treat some patients.

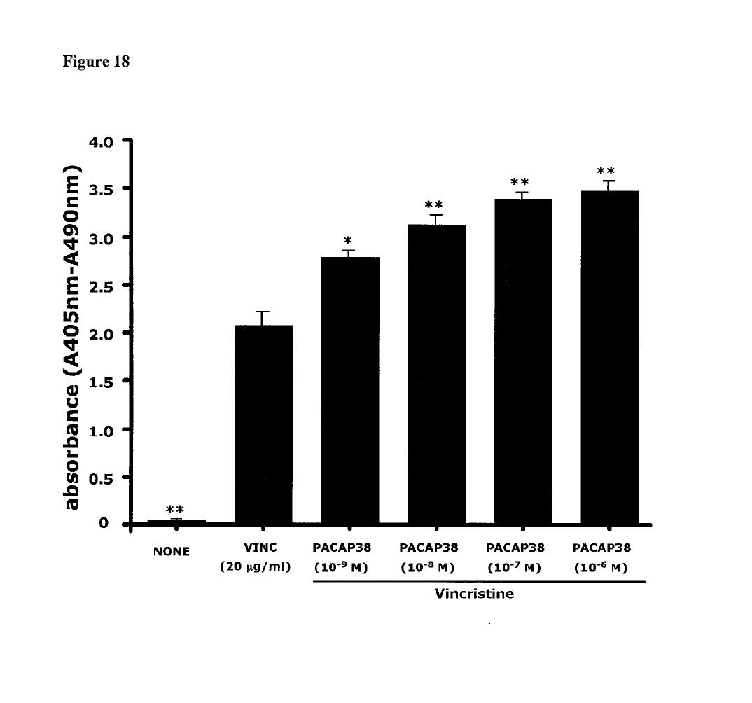

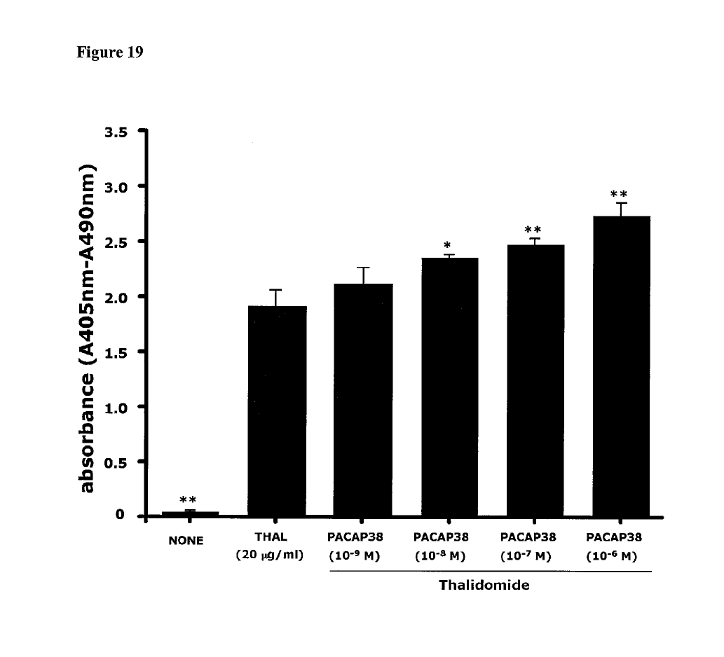

Treatment of human myeloma cells with either vincristine or thalidomide resulted in a large significant increase in apoptotic cell death (FIGS. 18 and 19). The addition of PACAP38 to the medium resulted in a dose-dependent enhancement of both vincristine-induced and thalidomide-induced apoptotic cell death of the human myeloma cells. PACAP38 significantly enhanced the vincristine-induced and the thalidomide-induced apoptotic cell death of these malignant plasma cells at doses as low as 10⁻⁹ M and 10⁻⁸ M, respectively.

### Example 10

### Enhancement of Thalidomide-Induced Apoptotic Cell Death of Erythroleukemia Cells by PACAP38 and PACAP Analogs

Myeloproliferative disorders are a diverse group of myeloid cancers that include (but are not limited to) myelofibrosis, myelodysplastic syndrome, chronic myelogenous leukemia, acute myelogenous leukemia, polycythemia vera, and essential thrombocytosis. Erythroleukemia is a rare form of myeloid leukemia. Drugs commonly used to treat myeloproliferative disorders include (but are not limited to) busulfan, hydroxyurea, doxorubicin, idarubicin, cytarabine, thalidomide, lenalidomide, vincristine, and imatinib mesylate (ST1571, Gleevec).

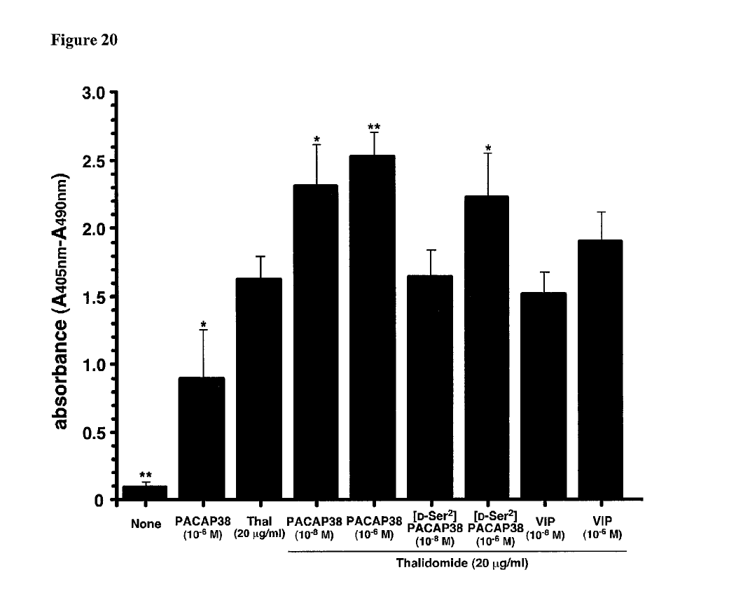

Treatment of human erythroleukemia cells with thalidomide resulted in a large significant increase in apoptotic cell death (FIG. 20). The addition of PACAP38, [D-Ser²]PACAP38 or VIP to the medium resulted in a dose-dependent enhancement of thalidomide-induced apoptotic cell death of the human erythroleukemia cells. PACAP38 significantly enhanced the thalidomide-induced apoptotic cell death of these malignant myeloid cells at doses as low as 10⁻⁸ M.

The above examples show that PACAP and PACAP analogs potently protect many types of cells and organs of the body against the toxic effects of several different classes of commonly used cancer chemotherapeutics (FIGS. 3-12). However, the above examples also show that PACAP and PACAP analogs protect non-hematopoietic cancer cells, such as pheochromocytoma and breast cancer cells, in vitro against the same anticancer agents (FIGS. 13 and 14). The latter observation is consistent with the published reports by others that PACAP(6-38), a PACAP/VIP receptor antagonist, inhibits the in vivo growth of xenografts of non-hematopoietic cancer cells such as human prostate, human breast and human non-small cell lung cancer cells, as solid tumors. On the other hand, the above examples show that PACAP and PACAP analogs enhanced the in vitro therapeutic effect of several anticancer agents on hematopoietic cancer cells, including both lymphoid (FIGS. 16-19) and myeloid (FIG. 20) cancer cells, and would be useful adjunctive therapeutics in combination with many commonly-used cancer chemotherapeutics for the treatment of hematological malignancies. The above examples show that there is a correlation between whether PACAP-like compounds stimulate or inhibit (FIG. 2) the rate of proliferation of some cancer cells and whether PACAP-like compounds reduce or enhance (FIGS. 16-20) the anticancer activity of some chemotherapeutics on some cancer cells.

### EQUIVALENTS

Those skilled in the art will recognize, or be able to ascertain using no more than routine experimentation, many equivalents to the specific embodiments of the present invention described herein. Such equivalents are intended to be encompassed by the following claims.

All publications, patents and patent applications mentioned in this specification are herein incorporated by reference into the specification to the same extent as if each individual publication, patent or patent application was specifically indicated to be incorporated herein by reference.

Other embodiments are in the claims.

What is claimed is:

1. A method for treating, reducing, or inhibiting an injury to an organ of a subject treated with an anticancer agent comprising administering to said subject an effective amount of a PACAP-like compound, wherein said PACAP-like compound treats, reduces, or inhibits said injury.

2. The method of claim 1, wherein said subject is a human.

3. The method of claim 1, wherein said PACAP-like compound is capable of binding to a PACAP/VIP receptor.

4. The method of claim 1, wherein said PACAP-like compound is selected from the group consisting of SEQ ID NOs:1-70.

5. The method of claim 4, wherein said PACAP-like compound comprises an N-acetyl group or a polyethylene glycol polymer with a molecular weight from about 4 kilodaltons to about 40 kilodaltons or wherein said PACAP-like compound is unamidated.

6. (canceled)

7. The method of claim 1, wherein said. PACAP-like compound is a Lys³⁸-propylamide derivative of a compound selected from the group consisting of SEQ ID NOs:1, 4-36, and 67-69, a Leu²⁷-propylamide derivative of a compound selected from the group consisting of SEQ ID NOs:2 and 37-56, an Asn²⁸-propylamide derivative of a compound selected from the group consisting of SEQ ID NOs:3 and 57-66, a peptidomimetic analog of a compound selected from the group consisting of SEQ ID NOs:1-70, or is SEQ ID NO: 70 or an Arthropod ortholog thereof.

8. The method of claim 7, wherein said PACAP-like compound further comprises an N-acetyl group.

9.-13. (canceled)

14. The method of claim 5, wherein said unamidated PACAP-like compound is flanked by amino-acid consensus sequences for one or more proteolytic enzymes.

15.-17. (canceled)

18. The method of claim 1, wherein said PACAP-like compound is administered to achieve a concentration of 10⁻¹⁴ M to 10⁻⁶ M in the blood of said subject or is administered to said subject at a dose of 1 μg to 1 gram.

19.-22. (canceled)

23. The method of claim 1, wherein said PACAP-like compound is administered by intravenous infusion, intraperitoneal injection, subcutaneous injection, or intramuscular injection, as an aerosol, intranasally, or orally.

24. (canceled)

25. The method of claim 1, wherein said injury to said organ is due to treatment of said subject with said anticancer agent, wherein said anticancer agent is selected from the group consisting of cisplatin, carboplatin, oxaliplatin, bleomycin, mitomycin C, calicheamicins, maytansinoids, doxorubicin, idarubicin, daunorubicin, epirubicin, busulfan, carmustine, lomustine, semustine, thalidomide, lenalidomide, methotrexate, 6-mercaptopurine, fludarabine, 5-azacytidine, pentostatin, cytarabine, gemcitabine, 5-fluorouracil, hydroxyurea, etoposide, teniposide, topotecan, irinotecan, chlorambucil, cyclophosphamide, ifosfamide, melphalan, bortezomib, vincristine, vinblastine, vinorelbine, paclitaxel, and docetaxel.

26. The method of claim 1, wherein said organ is the nervous system, the brain, the heart, the lung, the kidney, the liver, the pancreas, the gall bladder, the gastrointestinal tract, the adrenal gland, the thymus, the spleen, the lymph nodes, the breast, the ovary, the testes, or the prostate.

27.-40. (canceled)

41. The method of claim 1, wherein said subject is administered a viral vector encoding said PACAP-like compound or a cell that has been transfected with one or more polynucleotide sequences encoding said PACAP-like compound.

42. (canceled)

43. The method of claim 1, wherein said PACAP-like compound is administered i) a controlled release or a sustained release formulation, ii) after encapsulation in liposomes or microparticles, or iii) transcutaneously after encapsulation in dendrimers.

44.-45. (canceled)

46. The method of claim 1, wherein said PACAP-like compound is administered in combination with one or more cytoprotective adjunctive agents selected from amifostine, dexrazoxane, mesna, palifermin, and N-acetylcysteine.

47. (canceled)

48. The method of claim 1, wherein said anticancer agent is targeted preferentially to cancer cells by reversible conjugation to a monoclonal antibody or to one or more bioactive peptides.

49. The method of claim 1, wherein said PACAP-like compound reduces the incidence of delayed secondary cancers caused by said anticancer agent.

50.-51. (canceled)

52. The method of claim 1, wherein said subject has an epithelial cell cancer or a hematopoietic cancer, wherein said hematopoietic cancer is selected from a lymphoid cancer, a myeloid cancer, a lymphoid or myeloid leukemia, a B- or T-cell lymphoma, a plasma cell dyscrasia, or multiple myeloma.

53. The method of claim 52, wherein said organ has a dose-limiting toxicity to said anticancer agent and said PACAP-like compound is delivered to said organ of said subject.

54.-63. (canceled)

64. The method of claim 1, wherein said PACAP-like compound is administered to said subject prior to, after, or substantially simultaneously with, administration of said anticancer agent.

65.-66. (canceled)

67. The method of claim 1, wherein said PACAP-like compound is administered to said subject prior to or after said injury.

68.-70. (canceled)
